(19) United States
(12) Patent Application Publication
(10) Pub. No.: US 2010/0316670 A1
(43) Pub. Date: Dec. 16, 2010

(54) CHIMERIC SLE/DENGUE TYPE 4 ANTIGENIC VIRUSES

(76) Inventors: Joseph E. Blaney, Gettysburg, PA (US); Brian R. Murphy, Bethesda, MD (US); Alexander G. Pletnev, Gaithersburg, MD (US); Stephen S. Whitehead, Montgomery Village, MD (US)

Correspondence Address: OTT-NIH;C/O EDWARDS ANGELL PALMER & DODGE LLP, PO BOX 55874, BOSTON, MA, 02205, US

(21) Appl. No.: 12/664,293

(22) PCT Filed: Jun. 10, 2008
(86) PCT No.: PCT/US08/66445

§ 371 Date: Aug. 27, 2010

Related U.S. Application Data
(60) Provisional application No. 60/934,730, filed on Jun. 14, 2007.

(51) Int. Cl. A61K 39/295; C07H 21/00; C07K 14/08; C12N 7/01; A61P 37/04; A61P 31/14

(52) U.S. Cl. 424/202.1; 536/23.72; 530/350; 435/235.1

(57) ABSTRACT

Embodiments described herein concern attenuated, St. Louis Encephalitis Virus/dengue virus type 4 antigenic chimeric viruses, which can be used to prepare immunogenic compositions, vaccines, and diagnostic reagents. Methods of making and using the foregoing are provided.

### SEQUENCE LISTING

The present application is being filed along with a Sequence Listing in electronic format. The Sequence Listing is provided as a file entitled NIH361_{—}001VPC_Sequence_Listing.TXT, created Jun. 10, 2008, which is 208 Kb in size. The information in the electronic format of the Sequence Listing is incorporated herein by reference in its entirety.

### FIELD OF THE INVENTION

Aspects of the present invention concern attenuated, St. Louis Encephalitis Virus/dengue virus type 4 antigenic chimeric viruses, which can be used to prepare immunogenic compositions, vaccines, and diagnostic reagents. Methods of making and using the foregoing are also provided.

### BACKGROUND OF THE INVENTION

St. Louis encephalitis virus (SLE), a mosquito-borne flavivirus, is a member of the Japanese encephalitis virus (JE) serocomplex, which also includes West Nile virus (WN) and Murray Valley encephalitis virus [1]. SLE was first isolated in 1933 from the brain of a deceased patient during an outbreak of approximately 1,000 cases of encephalitis in St. Louis Mo. [2]. More than 10,000 cases of severe disease have been described since then in the United States of America, and the virus remains endemic throughout most of the country. The most recent extensive outbreak of disease associated with SLE was in 1990 when over 200 cases with 14 deaths were reported in central Florida [3]. SLE is also endemic in Central and South America, and recent reports have indicated increased human disease associated with SLE in Brazil and Argentina [4, 5].

Like WN, SLE is maintained in a natural transmission cycle between birds and Culex mosquitoes, and humans typically serve as incidental hosts [6]. High seroprevalence in sentinel chickens and positivity in mosquitoes during monitoring often predicts epidemic SLE human disease, as was the case in Florida in 1990. The ability to predict an increased period of SLE transmission indicates that a window of opportunity usually exists to vaccinate susceptible individuals. Human infection with SLE results in a spectrum of disease including asymptomatic infection, a general febrile illness, and potentially fatal meningitis/encephalitis [7]. The incidence of symptomatic to asymptomatic infection is reported to be approximately 1 to 300, although the elderly have a much greater risk of developing severe disease. Currently, a licensed vaccine is not available for prevention of SLE disease and the need for attenuated, yet immunogenic flaviviruses to be used in the development of vaccines for SLE is manifest.

### SUMMARY OF THE INVENTION

Aspects of the invention concern the development of two antigenic chimeric viruses, SLE/DEN4 (SEQ. ID. NOS. 5 and 6) and SLE/DEN4Δ30 (SEQ. ID. NOS. 7 and 8), which were generated by replacing the membrane precursor and envelope protein genes of dengue virus type 4 (DEN4) with those from St. Louis encephalitis virus (SLE) with or without a 30 nucleotide deletion in the DEN4 3′ untranslated region of the chimeric genome. Chimeric viruses were compared with parental wild-type SLE for level of neurovirulence and neuroinvasiveness in mice and for safety, immunogenicity, and protective efficacy in rhesus monkeys. The resulting viruses, SLE/DEN4 and SLE/DEN4Δ30, had greatly reduced neuroinvasiveness in immunodeficient mice but retained neurovirulence in suckling mice. Chimerization of SLE with DEN4 resulted in only moderate restriction in replication in rhesus monkeys, whereas the presence of the Δ30 mutation led to over-attenuation. Introduction of previously described attenuating paired charge-to-alanine mutations in the DEN4 NS5 protein of SLE/DEN4 reduced neurovirulence in mice and replication in rhesus monkeys. The two modified SLE/DEN4 viruses, SLE/DEN4-436,437 clone 641 (SEQ. ID. NOS. 9 and 10) and SLE/DEN4-654,655 clone 646 (SEQ. ID. NOS. 13 and 14), were found to have significantly reduced neurovirulence in mice and conferred protective immunity in monkeys against SLE challenge. Additional embodiments include the virus SLE/DEN4 551 (SEQ. ID. NOS. 11 and 12).

The working examples that follow describe the generation of the SLE/DEN4 and SLE/DEN4Δ30 constructs by replacing the prM and E protein genes of DEN4 or DEN4Δ30 with those of the Hubbard strain of SLE. Antigenic chimerization of SLE with DEN4 or DEN4Δ30 resulted in greatly reduced neuroinvasiveness for severe combined immunodeficient (SCID) mice but no reduction in neurovirulence for immunocompetent mice. SLE/DEN4 was moderately attenuated and immunogenic in rhesus monkeys while SLE/DEN4Δ30 was over-attenuated. Further attenuation the SLE/DEN4 antigenic chimeric virus was achieved by introducing paired charge-to-alanine attenuating mutations in the nonstructural NS5 protein of SLE/DEN4. The resulting SLE/DEN4 mutants exhibited reduced neurovirulence in mice and provided complete protection in rhesus monkeys against challenge with SLE.

Accordingly, several embodiments include the chimeric virus or nucleic acids encoding said viruses described herein (e.g., SEQ. ID. NOS.: 5, 6, 7, 8, 9, 10, 11, 12, 13, or 14, including ATCC deposits ______) and methods of making and using these compositions to generate immunogens and vaccines for the induction of an immune response and the establishment of protection in a subject. Some embodiments concern a nucleic acid comprising a first nucleotide sequence encoding at least one structural protein from a SLE virus and a second nucleotide sequence encoding at least one nonstructural protein from a dengue virus. In some embodiments, said at least one non-structural protein from a dengue virus comprises at least NS5 and in some embodiments said NS5 protein comprises at least one mutation. Preferably, said at least one mutation is a substitution at position 654 or 655 or a corresponding position when said dengue virus is not DEN4 or DEN4Δ30 and said at least one structural protein from a SLE virus is the E protein. Said E protein may also comprise at least one mutation and said at least one mutation in the E protein is preferably a substitution at position 156 or a corresponding position when said SLE virus is not the Hubbard strain of SLE. Thus, several embodiments concern the nucleic acids above, wherein said E protein comprises at least one mutation and said at least one mutation in the E protein is at position 156 or a corresponding position when said SLE virus is not the Hubbard strain of SLE. In these embodiments, the dengue virus portion of the construct may be dengue type 1 virus, dengue type 2 virus, dengue type 3 virus, or dengue type 4 virus.

In some aspects of the invention, the nucleic acids above comprises a dengue virus that is an attenuated virus or a virus adapted for enhanced replication in Vero cells. The dengue virus can be a dengue type 4 virus and the virus can be attenuated by a deletion of about 30 nucleotides from the 3′ untranslated region of the dengue type 4 genome corresponding to the TL2 stem-loop structure between about nucleotides 10478-10507. Further embodiments include a nucleic acid as described above, wherein the dengue virus is dengue type 1 virus and the virus is attenuated by a deletion of about 30 nucleotides from the 3′ untranslated region of the dengue type 1 genome corresponding to the TL2 stem-loop structure between about nucleotides 10562-10591. More embodiments include a nucleic acid as described above, wherein the dengue virus is dengue type 2 virus and the virus is attenuated by a deletion of about 30 nucleotides from the 3′ untranslated region of the dengue type 2 genome corresponding to the TL2 stem-loop structure between about nucleotides 10541-10570. Still more embodiments include a nucleic acid as described above, wherein the dengue virus is dengue type 3 virus and the virus is attenuated by a deletion of about 30 nucleotides from the 3′ untranslated region of the dengue type 3 genome corresponding to the TL2 stem-loop structure between about nucleotides 10535-10565.

In some embodiments, the nucleic acids above include a first nucleotide sequence that encodes at least two structural proteins from a SLE virus, In some embodiments, the nucleic acids above comprise structural proteins from a SLE virus, such as prM and E proteins. Preferably, the nucleic acids above comprise, consist, or consist essentially of a nucleic acid that is selected from the group consisting of SEQ. ID. NOs.: 5, 7, 9, 11, and 13 or a fragment thereof, which contains at least a first nucleotide sequence encoding at least one structural protein from a SLE virus and a second nucleotide sequence encoding at least one nonstructural protein from a dengue virus.

Aspects of the invention also include a polypeptide encoded by any one or more of the nucleic acids above. Preferably said polypeptide comprises, consists or consists essentially of SEQ. ID. NOS.: 6, 8, 10, 12, or 14. Embodiments also include a chimeric virus comprising any one or more of the nucleic acids or polypeptides described above. More embodiments include an immunogenic composition comprising any one or more of the nucleic acids or polypeptides or the chimeric viruses described above. In some aspects of the invention, the immunogenic compositions are used to induce an immune response. That is, some embodiments include a method of inducing an immune response in a subject comprising providing an effective amount of one or more of the compositions above to the subject and measuring the induction of an immune response in said subject.

More embodiments concern a vaccine comprising any one or more of the nucleic acids or polypeptides or the chimeric viruses described above. Accordingly some embodiments concern a method of providing protection against SLE infection in a subject comprising identifying a subject in need of protection against SLE infection and providing an effective amount of one or more of the compositions above to the subject.

### BRIEF DESCRIPTION OF THE DRAWINGS

FIG. 1 shows the molecular construction of recombinant SLE/DEN4 antigenic chimeric viruses. (A) The prM/E structural protein region of the DEN4 cDNA plasmid p4 [37] was replaced with the corresponding region from SLE Hubbard to generate the SLE/DEN4 virus. The Δ30 deletion, a 30 nucleotide deletion in the 3′UTR (DEN4 nucleotides 10,478-10,507), was introduced into the SLE/DEN4 cDNA plasmid and was used to recover the SLE/DEN4Δ30 virus. The amino acid sequences surrounding the C/prM junctions at the protease cleavage site of parental and chimeric viruses are indicated. The P1′-P4′ amino acids of the resulting C/prM cleavage junctions in the SLE/DEN4 viruses are indicated in bold font. Arrows indicate the putative cleavage site at the C/prM junction mediated by the NS2B-NS3 protease. (B) The eight pairs of charge-to-alanine mutations introduced into the DEN4 NS5 protein in SLE/DEN4 are indicated. The numbering indicates the position of the amino acid (a.a.) pair within the NS5 protein. Each individual wild type pair of amino acids was mutated to a pair of alanines in the SLE/DEN4 cDNA clone to generate eight modified SLE/DEN4 viruses. Two independent virus clones were recovered for each mutation and are identified by a 3 digit clone number; e.g. SLE/DEN4-22,23 clones 653 and 654.

FIG. 2 shows the neurovirulence of the modified SLE/DEN4 viruses containing NS5 mutations. Litters of approximately ten three-day-old Swiss Webster mice were inoculated IC with 10² PFU of indicated virus. Mice were monitored for signs of encephalitis and morbidity for 21 days and the percent survival for each group is indicated. Two virus clones for each modified SLE/DEN4 virus were included in the study.

FIG. 3 shows the replication of parental and chimeric viruses in mouse brain. Five-day-old Swiss Webster mice were inoculated IC with 10³ PFU of indicated virus. Brains were removed on odd days post-infection from four mice per group and virus titer was determined by plaque assay in Vero cells. Mean peak virus titers (log₁₀PFU/g of brain) are indicated in parentheses. The limit of detection (10^{1.7} PFU/g) is indicated by a dashed line. The end of a data line prior to day 21 indicates that all mice had succumbed to infection. The values for DEN4 are historical data included for comparison [10].

FIG. 4 shows the growth analysis of parental SLE and chimeric viruses in Vero cells or in human neuroblastoma cells following incubation at different temperatures. An efficiency of plaque formation assay was performed with the indicated viruses in Vero cells and SH-SY5Y cells. Confluent monolayers of cells were infected with serial ten-fold dilutions of virus at 32° C., overlaid with semisolid growth media, and then incubated for five days at 32, 35, 36, 37, 38, or 39° C. Plaques were visualized by immunostaining and quantitated. The limit of detection (10^{0.7} PFU/ml) is indicated by a dashed line.

FIG. 5(A) shows the SLE/DEN4 plasmid consensus sequence (G-G-T-R JUNCTION); the start and stop codons are in bold and underlined and vector sequence is in lowercase and in bold. (B) shows the encoded polyprotein.

FIG. 6(A) shows the SLE/DEN4Δ30 VIRUS 545 virus sequence; the site of the Δ30 deletion mutation is marked by underline. (B) shows the encoded polyprotein.

FIG. 7(A) shows the SLE/DEN4-436,437 VIRUS 641 virus sequence; the site of the Phe₁₅₆→Ser mutation and the mutations at 436, 437 are marked by underline and bold. (B) shows the encoded polyprotein.

FIG. 8(A) shows the SLE/DEN4 VIRUS CLONE 551 virus sequence. (B) shows the encoded polyprotein.

FIG. 9(A) shows the SLE/DEN4-654,655 VIRUS CLONE 646 virus sequence; the 654,655 mutation is marked in bold and underlined. (B) shows the encoded polyprotein.

### DETAILED DESCRIPTION OF THE PREFERRED EMBODIMENTS

SLE and West Nile Virus (WN) are closely related with 70-75% amino acid homology in the E glycoprotein, which is the main target of neutralizing antibodies, and evidence exists for some cross-protection between the two viruses and other members of the JE serocomplex in birds, hamsters, and humans [12, 13]. A bivalent WN and SLE vaccine, however, will likely be required to induce long-term immunity to both viruses. The need for such a medicament has been long felt and the present inventors set-out to generate a live attenuated SLE component for such a bivalent vaccine.

Since the first reports of the generation of intratypic (DEN2/DEN4) and intertypic (TBE/DEN4) antigenic chimeric flaviviruses using reverse genetics [15, 22], numerous chimeric viruses have been created using genes from tick-borne and mosquito-borne flaviviruses, and many of these viruses are currently being evaluated as vaccines [10, 16, 19, 20, 23-26]. Recent clinical studies have indicated that antigenic chimeric flaviviruses are attenuated and immunogenic in human volunteers and may serve as live attenuated virus vaccines for protection against disease caused by DEN, JE, WN, and TBE viruses [27-31].

A live attenuated antigenic chimeric WN vaccine candidate, designated WN/DEN4Δ30, for example, was generated by replacing the pre-membrane (prM) and envelope (E) protein genes of DEN4Δ30 with those of WN strain NY99 [8]. WN/DEN4Δ30 was found to be attenuated in mice, monkeys, geese, and mosquitoes [9-11]. Two genetic factors contributed to attenuation of WN/DEN4Δ30: (1) antigenic chimerization led to reduced neuroinvasiveness and neurovirulence in mice and restricted replication in monkeys and (2) the presence of the Δ30 deletion mutation in the 3′ untranslated region (UTR) at nucleotides 10,478-10,507 further attenuated WN/DEN4 for monkeys [9-11]. WN/DEN4Δ30 replicated to a peak titer of between 10^{3.4} and 10^{4.9} PFU/g of brain after intracerebral inoculation of suckling mice; whereas, WN reached a peak titer of nearly 10¹² PFU/g of brain indicating that the chimeric virus has greatly reduced neurovirulence. In rhesus monkeys, viremia was not detectable during WN/DEN4Δ30 infection, but a strong neutralizing antibody response was induced that conferred protection from wild type WN infection. A phase I clinical trial of the WN/DEN4Δ30 vaccine candidate is in progress.

Attenuation of antigenic chimeric flaviviruses for mice, mosquitoes, or non-human primates can result from two different mechanisms. First, the simple construction of an antigenic chimeric virus from two wild-type parent viruses can yield a virus that has either reduced virulence or a restricted level of replication compared with either parent virus. For example, the DEN2/DEN4 and the DEN3/DEN4 chimeric viruses were found to be attenuated in rhesus monkeys and mosquitoes without the introduction of any specific attenuating mutations [20, 32]. The WN/DEN4 and the LGT/DEN4 chimeric viruses were also attenuated for neurovirulence and neuroinvasiveness for mice and for replication in monkeys [8-10, 16, 21]. In these examples, antigenic chimerization led to attenuation of both neuroinvasiveness and neurovirulence in mice for the neurotropic viruses or to restricted replication in rhesus monkeys for each of the four viruses. However, the TBE/DEN4 chimeric virus exhibited greatly reduced neuroinvasiveness but retained a high level of neurovirulence for mice indicating that chimerization in this case did not result in a decrease in virulence for the murine CNS [19, 33]. In addition, the TBE/DEN4 chimeric virus replicated efficiently in rhesus monkeys [19].

As described in the examples that follow, SLE/DEN4 (SEQ. ID. NO. 5) and SLE/DEN4Δ30 (SEQ. ID. NO. 7) constructs were generated by replacing the prM and E protein genes of DEN4 or DEN4Δ30 with those of the Hubbard strain of SLE. The present inventors contemplated that the chimerization of SLE with DEN4 would result in a pattern of neuroinvasiveness and neurovirulence in mice and replication in monkeys that was similar to that of its closest relative, WN, but this did not turn out to be the case. Rather, SLE/DEN4 resembled the TBE/DEN4 chimeric virus exhibiting diminished neuroinvasiveness while retaining a high level of neurovirulence in mice. SLE/DEN4, like TBE/DEN4, exhibited only moderate attenuation in rhesus monkeys. These results indicate that antigenic chimerization routinely yields vaccine candidates that have restricted neuroinvasiveness for mice, but does not always yield chimeric viruses with a predictable level of mouse neurovirulence or restricted replication in rhesus monkeys. Thus, the mechanism of the attenuation afforded by the incompatibility of antigenic chimeric virus proteins appears to be governed by highly virus-specific genetic elements whose in vivo effects cannot be predicted by genetic or antigenic relatedness.

The second mechanism that leads to attenuation of antigenic chimeric viruses is the presence of attenuating mutations either in the genetic background of the antigenic chimeric virus [19, 34] or in the prM or E protein [33, 35]. Several attenuated flaviviruses have been utilized to generate vaccine candidates including the yellow fever vaccine virus [35], a DEN2 virus that has been attenuated by serial in vitro passage [36], and, as described herein, a DEN4 virus attenuated by a deletion (the Δ30 deletion mutation) in the 3′UTR [37]. The Δ30 deletion mutation in the DEN4 virus has been shown to be highly attenuating and genetically stable in humans and, as such, has been selected for inclusion in antigenic chimeric viruses [38]. Addition of the Δ30 deletion mutation to the DEN2/DEN4 [20] and the DEN3/DEN4 [32] chimeric viruses did not further attenuate the virus for rhesus monkeys, but the mutation had a highly significant attenuating effect on TBE/DEN4 [19] and WN/DEN4 [9, 10] in rhesus monkeys.

As shown in the examples below, addition of the Δ30 deletion mutation to SLE/DEN4 over-attenuated the virus for rhesus monkeys resulting in no detectable serum neutralizing antibodies after immunization and insufficient protection from SLE challenge. Thus, the Δ30 deletion mutation was highly attenuating in both WN/DEN4 and SLE/DEN4, but the level of attenuation for SLE/DEN4 was sufficiently high that it rendered the chimeric virus poorly immunogenic in rhesus monkeys, and, therefore it was not useful as a vaccine for SLE. A set of at least three properties that identify an antigenic chimeric virus as suitable for evaluation in humans include: (1) evidence of decreased viremia in rhesus monkeys; (2) ability to induce a protective immune response in monkeys; and (3) reduced neurovirulence in mice since some viruses that exhibit significant neurovirulence for mice can retain neurovirulence for the CNS of primates [39].

Since SLE/DEN4 was only moderately attenuated and SLE/DEN4Δ30 was over-attenuated in rhesus monkeys and both viruses retained neurovirulence for mice, the present inventors set-out to identify additional mutations that could further attenuate SLE/DEN4. To potentially achieve a reduction in the neurovirulence of SLE/DEN4 for mice, paired charge-to-alanine mutations that were previously shown to reduce replication of rDEN4 in the mouse brain were introduced into the SLE/DEN4 chimeric virus [14]. Two mutations, NS5 Asp₆₅₄Arg₆₅₅→AlaAla and E Phe₁₅₆→Ser, were identified that conferred one or more of the three desirable properties outlined above. The NS5 Asp₆₅₄Arg₆₅₅→AlaAla mutation, which has a strong ts phenotype, conferred reduced neurovirulence and reduced replication in the brain of suckling mice inoculated with SLE/DEN4-654,655. SLE/DEN4-654,655 manifested an approximately 1,000-fold reduction in peak virus titer and an eight day delay in attaining peak virus titer when compared with mice infected with SLE. However, this reduction was less than that exhibited by WN/DEN4Δ30 [10].

Importantly, SLE/DEN4-654,655 was highly restricted in replication in rhesus monkeys as no monkey had detectable viremia, indicating that the introduction of the 654,655 mutation into SLE/DEN4 further attenuated this virus for both rhesus monkeys and for the CNS of mice. Although SLE/DEN4-654,655 was only weakly immunogenic in rhesus monkeys, immunization with it provided complete protection against replication of SLE challenge virus in the monkeys. Thus, this mutation achieved each of the three desired properties of an acceptable SLE vaccine candidate. Since SLE/DEN4-654,655 is a temperature sensitive virus and since rhesus monkeys have a higher core body temperature (39° C.) than humans (37° C.), it is possible that SLE/DEN4-654,655 will replicate to a greater extent in humans than in rhesus monkeys and thereby will induce a higher level of neutralizing antibodies in the human host. The NS5 Asp₆₅₄Arg₆₅₅→AlaAla mutation contains 4 nucleotide substitutions and is expected to exhibit greater genetic and phenotypic stability than that of a virus with a single point mutation, but this requires experimental verification.

An adventitious mutation, Phe₁₅₆→Ser in the E glycoprotein, developed independently in three SLE/DEN4 chimeric viruses, each of which manifested greatly reduced neurovirulence in mice. The association of the mutation in three separate viruses with reduced neurovirulence in mice suggests a causal relationship. SLE/DEN4-436,437 clone 641, which contained this mutation, was highly attenuated and greatly restricted in replication in the CNS of mice whereas another clone of this virus lacking the Phe₁₅₆→Ser mutation retained neurovirulence. SLE/DEN4-436,437 clone 641 exhibited over a 400,000-fold reduction in peak titer compared to that of SLE. However, SLE/DEN4-436,437 clone 641 was not more attenuated than SLE/DEN4 in rhesus monkeys. As expected, it was immunogenic and provided protection against challenge. The reduced neurovirulence presumably mediated by Phe₁₅₆→Ser in E is conferred by a single nucleotide substitution and therefore may be susceptible to genetic instability or reversion. A construct encoding a recombinant virus with an engineered E Phe₁₅₆→Ser mutation will confirm the in vivo effects of this mutation and such a construct is contemplated for use in several embodiments described herein.

Since the E Phe₁₅₆→Ser mutation was not present in the cDNA clones used to recover each of the three SLE/DEN viruses (clones 549, 554, and 641), it was contemplated that that the chimeric viruses bearing this adventitious mutation replicate more efficiently in Vero cells and were independently selected during the cloning process. Analysis of the E Phe₁₅₆→Ser locus indicated that this amino acid change would result in a putative N-linked glycosylation site at amino acids 154-156 (Asn-Tyr-Phe→Asn-Tyr-Ser) that is present in other SLE virus strains [40].

As described herein, suitable attenuated and immunogenic vaccine candidates for SLE can be combined with WN vaccine candidates so as to generate a bivalent vaccine. Preferred WN vaccine candidates include WN/DEN4Δ30. Such a bivalent vaccine will provide protection from the two neurotropic, mosquito-borne flaviviruses endemic in the United States and is particularly useful for the inoculation of at-risk groups including the elderly. The following section describes in greater detail several approaches to generate a flavivirus chimera, which will induce an immune response to SLE and or provide protection against SLE challenge or infection.

Flavivirus Chimeras, Immunogenic Compositions, and Vaccines

Immunogenic SLE/DEN flavivirus chimeras and methods of making and using these compositions are provided in this section. It is contemplated that the SLE flavivirus chimeras generated as described herein can be incorporated into immunogenic compositions and vaccines by themselves (e.g., “neat” formulations) with or without additional elements such as, carriers, adjuvants, or immunomodulating molecules. These compositions can be used to induce an immune response to SLE and/or to provide protection against SLE challenge or infection. Additionally, the immunogenic compositions and vaccines containing the SLE flavivirus chimeras can, optionally, include West Nile virus (WN) immunogenic compositions or vaccines so as to form a bivalent immunogenic composition or vaccine that would induce an immune response to and/or provide protection from the two mosquito-borne flaviviruses that are endemic in the US. Exemplary WN immunogenic compositions, vaccines, and WN chimeras that are suitable for the preparation of such bivalent compositions are disclosed in U.S. Pat. Pub. No. 20050100886, herein expressly incorporated by reference in its entirety. Furthermore, the compositions described herein can be used to generate diagnostic reagents, such as monoclonal or polyclonal antibodies, which may be incorporated into diagnostic kits, therapeutics or preventative medicaments.

Several of the compositions described herein comprise nucleotide sequences that encode an SLE viral immunogenic protein or antigenic fragment thereof and further nucleotide sequences selected from the backbone of a dengue virus (e.g., dengue virus, type 4), which may be modified by mutation so as to reduce or attenuate neurovirulence and/or neuroinvasiveness. These nucleotide sequences and/or the chimeric viruses derived therefrom can be incorporated into an immunogenic composition or vaccine and these compositions can be provided to subjects that have been identified as ones in need of an immune response to SLE so as to induce an immune response and/or to confer protection in said subjects against SLE infection. That is, the nucleic acids encoding said chimeric viruses can be provided to said subjects in the form of a DNA vaccine or immunogenic composition, for example, so as to induce an immune response to SLE (e.g., increase in IgG titer or T cell response) or to provide protection against SLE infection. Optionally, the chimeric viruses can be provided to said subjects so as to induce an immune response to SLE (e.g., increase in IgG titer or T cell response) or provide protection against SLE infection.

Subjects in need of an immune response to SLE and/or protection against SLE infection can be identified by diagnostic methods and/or clinical evaluation. Additionally, any one or more of the compositions described herein can be provided to a subject thought to be at risk of acquiring SLE (e.g., individuals visiting mosquito-infested regions). In this instance, the subject can be said to be identified as one in need of an immune response to SLE and/or protection against SLE infection. Accordingly, aspects of the invention concern methods of inducing an immune response against SLE (e.g., increase in IgG titer or T cell response or both) and/or providing protection against SLE virus challenge (e.g., protection from SLE infection) comprising the steps of identifying a subject in need of an immune response against SLE (e.g., increase in IgG titer or T cell response or both) and/or in need of protection against SLE virus challenge (e.g., protection from SLE infection) and providing said subject any one or more of the compositions described herein (e.g., a composition comprising an SLE/Den4 chimera or a nucleic acid encoding said SLE/DEN4 chimera). Optionally, the methods described herein may include a measuring step, wherein the immune response to SLE in a subject or an assay is measured prior to, during, or after providing the SLE/DEN4 chimera or nucleic acid encoding said SLE/DEN4 chimera. The measurement can take the form of measuring antibody generated to an SLE antigen, measuring SLE viral titer or the reduction thereof, measuring a T cell response or simply observing clinical or health benefit or patient improvement after receiving a composition described herein.

In some embodiments, the preferred SLE/DEN chimera is generated from a nucleic acid that comprises a first nucleotide sequence encoding at least one protein from a SLE virus (e.g., the C, prM, and/or E proteins), and a second nucleotide sequence encoding a nonstructural protein from a dengue virus (e.g., DEN4). In some embodiments, the dengue virus is attenuated.

As used herein, in some contexts, the term “residue” can refer to an amino acid (D or L) or an amino acid mimetic that is incorporated into a peptide by an amide bond. As such, the amino acid may be a naturally occurring amino acid or, unless otherwise limited, may encompass known analogs of natural amino acids that function in a manner similar to the naturally occurring amino acids (i.e., amino acid mimetics). Moreover, an amide bond mimetic includes peptide backbone modifications well known to those skilled in the art.

Furthermore, as one of skill in the art will readily appreciate certain individual substitutions, deletions or additions to the amino acid sequence of a composition described herein, or in the nucleotide sequence encoding for the amino acids of a sequence of one of the compositions described herein, will have very little if any functional impact on the composition. Accordingly, in this instance, said compositions containing these types of non-effectual mutations are considered to be equivalent to the compositions described herein. In some circumstances, these non-effectual mutations may be the result of an alteration, addition, or deletion of a single amino acid or a small percentage of amino acids (typically less than 5%, more typically less than 1%) in an encoded sequence are conservatively modified variations, wherein the alterations result in the substitution of an amino acid with a chemically similar amino acid. Conservative substitution tables providing functionally similar amino acids are well known. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In some contexts, the term “virus chimera,” “chimeric virus,” “flavivirus chimera” and “chimeric flavivirus” are used interchangeably to refer to an infectious construct generated as described herein that comprises a nucleotide sequence encoding the immunogenicity of a SLE virus and further nucleotide sequences derived from the backbone of a dengue virus (e.g., DEN4).

Additionally, the term “infectious construct” can refer to a virus, a viral construct, a viral chimera, a nucleic acid derived from a virus or any portion thereof, which may be used to infect a cell in certain contexts and the term “nucleic acid chimera” can refer to a construct generated as described herein, wherein said construct comprises a nucleic acid that encodes an immunogenic portion or fragment or protein of the SLE virus and further nucleotide sequences derived from the backbone of a dengue virus (e.g., DEN4). Correspondingly, any chimeric flavivirus or flavivirus chimera generated as described herein can be recognized as an example of a nucleic acid chimera in the appropriate context.

The flavivirus chimeras described herein can be produced by substituting at least one of the structural protein genes of the SLE virus against which immunity is desired into a dengue virus genome backbone, using recombinant engineering techniques, namely, by removing a designated dengue virus gene and replacing it with the desired corresponding gene of SLE virus. Alternatively, using the sequences provided in GenBank, the nucleic acid molecules encoding the flavivirus proteins may be synthesized using known nucleic acid synthesis techniques and inserted into an appropriate vector. Attenuated, immunogenic virus is therefore produced using recombinant engineering techniques known to those skilled in the art.

As mentioned above, the gene to be inserted into the backbone encodes a SLE structural protein. Preferably the SLE gene to be inserted is a gene encoding a C protein, a prM protein and/or an E protein. The sequence inserted into the dengue virus backbone can encode both the prM and E structural proteins. The sequence inserted into the dengue virus backbone can encode the C, prM and E structural proteins. The dengue virus backbone can be the DEN1, DEN2, DEN3, or DEN4 virus genome, or an attenuated dengue virus genome of any of these serotypes, and includes the substituted gene(s) that encode the C, prM and/or E structural protein(s) of a SLE virus or the substituted gene(s) that encode the prM and/or E structural protein(s) of a SLE virus. Preferably, the chimeric viruses comprise one or more mutations in the NS5 domain of the DEN backbone (e.g., DEN1, DEN2, DEN3, or DEN4), such as a mutation at position 654, or 655. Additionally, one or more chimeric viruses may have a mutation at position 156 of the E protein. Some chimeric viruses prepared as described herein, comprise an NS5 mutation (e.g., at position 654 or 655) and a mutation of the E protein at position 156.

Suitable chimeric viruses or nucleic acid chimeras containing nucleotide sequences encoding structural proteins of SLE virus can be evaluated for usefulness as vaccines by screening them for phenotypic markers of attenuation that indicate reduction in virulence with retention of immunogenicity. Antigenicity and immunogenicity can be evaluated using in vitro or in vivo reactivity with SLE antibodies or immunoreactive serum using routine screening procedures known to those skilled in the art.

The preferred chimeric viruses and nucleic acid chimeras provide live, attenuated viruses useful as immunogens or vaccines. In a preferred embodiment, the chimeras exhibit high immunogenicity while at the same time not producing dangerous pathogenic or lethal effects. The chimeric viruses or nucleic acid chimeras can comprise the structural genes of a SLE virus in a wild-type or an attenuated dengue virus backbone. For example, the chimera may express the structural protein genes of a SLE virus in either of a dengue virus or an attenuated dengue virus background.

The strategy described herein of using a genetic background that contains nonstructural regions of a dengue virus genome, and, by chimerization, the properties of attenuation, to express the structural protein genes of a SLE virus has lead to the development of live, attenuated flavivirus vaccine candidates that express structural protein genes of desired immunogenicity. Thus, vaccine candidates for control of SLE pathogens can be designed.

Viruses used in the chimeras described herein can be grown using various techniques. Virus plaque or focus forming unit (FFU) titrations are then performed and plaques or FFU are counted in order to assess the viability, titer and phenotypic characteristics of the virus grown in cell culture. Wild type viruses are mutagenized to derive attenuated candidate starting materials.

Chimeric infectious clones are constructed from various flavivirus strains. The cloning of virus-specific cDNA fragments can also be accomplished, if desired. The cDNA fragments containing the structural protein or nonstructural protein genes are amplified by reverse transcriptase-polymerase chain reaction (RT-PCR) from flavivirus RNA with various primers. Amplified fragments are cloned into the cleavage sites of other intermediate clones. Intermediate, chimeric flavivirus clones are then sequenced to verify the sequence of the inserted flavivirus-specific cDNA.

Full genome-length chimeric plasmids constructed by inserting the structural or nonstructural protein gene region of flaviviruses into vectors are obtainable using recombinant techniques well known to those skilled in the art. The next section provides greater detail on several approaches that can be used to administer or to provide one or more of the compositions described herein to a subject in need of an immune response to SLE.

Method of Administration

The viral chimeras described herein are individually or jointly combined with a pharmaceutically acceptable carrier or vehicle for administration as an immunogen or vaccine to humans or animals. The terms “pharmaceutically acceptable carrier” or “pharmaceutically acceptable vehicle” are used herein to mean any composition or compound including, but not limited to, water or saline, a gel, salve, solvent, diluent, fluid ointment base, liposome, micelle, giant micelle, and the like, which is suitable for use in contact with living animal or human tissue without causing adverse physiological responses, and which does not interact with the other components of the composition in a deleterious manner.

The immunogenic or vaccine formulations may be conveniently presented in viral plaque forming unit (PFU) unit or focus forming unit (FFU) dosage form and prepared by using conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers. Formulations suitable for parenteral administration include aqueous and non-aqueous sterile injection solutions, which may contain anti-oxidants, buffers, bacteriostats and solutes that render the formulation isotonic with the blood of the intended recipient, and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets commonly used by one of ordinary skill in the art.

Preferred unit dosage formulations are those containing a dose or unit, or an appropriate fraction thereof, of the administered ingredient. It should be understood that in addition to the ingredients particularly mentioned above, the formulations of the present invention may include other active or inactive agents. A variety of adjuvants may be administered in conjunction with a chimeric virus in the immunogen or vaccine composition described herein (e.g., co-administration or mixed with the composition). Such adjuvants include, but are not limited to, polymers, co-polymers such as polyoxyethylene-polyoxypropylene copolymers, including block co-polymers, polymer p 1005, Freund's complete adjuvant (for animals), Freund's incomplete adjuvant; sorbitan monooleate, squalene, CRL-8300 adjuvant, alum, QS 21, muramyl dipeptide, CpG oligonucleotide motifs and combinations of CpG oligonucleotide motifs, trehalose, bacterial extracts, including mycobacterial extracts, detoxified endotoxins, membrane lipids, iscoms, or combinations thereof.

The immunogenic or vaccine composition may be administered through different routes, such as oral or parenteral, including, but not limited to, buccal and sublingual, rectal, aerosol, nasal, intramuscular, subcutaneous, intradermal, and topical. The composition may be administered in different forms, including, but not limited to, solutions, emulsions and suspensions, microspheres, particles, microparticles, nanoparticles and liposomes. It is expected that from about 1 to about 5 doses may be required per immunization schedule. Initial doses may range from about 100 to about 100,000 PFU or FFU, with a preferred dosage range of about 500 to about 20,000 PFU or FFU, a more preferred dosage range of from about 1000 to about 12,000 PFU or FFU and a most preferred dosage range of about 1000 to about 4000 PFU or FFU. Booster injections may range in dosage from about 100 to about 20,000 PFU or FFU, with a preferred dosage range of about 500 to about 15,000, a more preferred dosage range of about 500 to about 10,000 PFU or FFU, and a most preferred dosage range of about 1000 to about 5000 PFU or FFU. For example, the volume of administration will vary depending on the route of administration. Intramuscular injections may range in volume from about 0.1 ml to 1.0 ml.

The composition may be stored at temperatures of from about −100° C. to about 4° C. The composition may also be stored in a lyophilized state at different temperatures including room temperature. The composition may be sterilized through conventional means known to one of ordinary skill in the art. Such means include, but are not limited to, filtration. The composition may also be combined with bacteriostatic agents to inhibit bacterial growth.

The immunogenic or vaccine composition described herein may be administered to humans or domestic animals, such as horses or birds, especially individuals travelling to regions where SLE infection is present, and also to inhabitants of those regions. The optimal time for administration of the composition is about one to three months before the initial exposure to the SLE virus. However, the composition may also be administered after initial infection to ameliorate disease progression, or after initial infection to treat the disease. The next section provides greater detail on additional embodiments, such as diagnostic reagents, which can incorporate or can be prepared from one or more of the compositions described herein.

Diagnostic and Biotechnological Tools

Aspects of the invention also include diagnostic reagents and biotechnological tools that include or are developed from one or more of the compositions described herein. Nucleic acid sequences of SLE virus and dengue virus are useful for designing nucleic acid probes and primers for the detection of SLE virus and dengue virus chimeras in a sample or specimen with high sensitivity and specificity, for example. Probes or primers corresponding to SLE virus and dengue virus can be used to detect the presence of an SLE virus in a subject. The nucleic acid and corresponding amino acid sequences are also useful as laboratory tools to study the organisms and diseases and to develop other therapies and treatments for the diseases.

Nucleic acid probes and primers selectively hybridize with nucleic acid molecules encoding SLE virus and dengue virus or complementary sequences thereof. By “selective” or “selectively” is meant a sequence which does not hybridize with other nucleic acids to prevent adequate detection of the SLE virus sequence and dengue virus sequence. Therefore, in the design of hybridizing nucleic acids, selectivity will depend upon the other components present in the sample. The hybridizing nucleic acid should have at least 70% complementarity with the segment of the nucleic acid to which it hybridizes. As used herein to describe nucleic acids, the term “selectively hybridizes” excludes the occasional randomly hybridizing nucleic acids, and thus has the same meaning as “specifically hybridizing.” The selectively hybridizing nucleic acid probes and primers of this invention can have at least 70%, 80%, 85%, 90%, 95%, 97%, 98% and 99% complementarity with the segment of the sequence to which it hybridizes, preferably 85% or more.

Aspects of the present invention also comprise sequences, probes and primers that selectively hybridize to the encoding nucleic acid or the complementary, or opposite, strand of the nucleic acid. Specific hybridization with nucleic acid can occur with minor modifications or substitutions in the nucleic acid, so long as functional species-species hybridization capability is maintained. By “probe” or “primer” is meant nucleic acid sequences that can be used as probes or primers for selective hybridization with complementary nucleic acid sequences for their detection or amplification, which probes or primers can vary in length from about 5 to 100 nucleotides, or preferably from about 10 to 50 nucleotides, or most preferably about 18-24 nucleotides. Isolated nucleic acids are provided herein that selectively hybridize with the species-specific nucleic acids under stringent conditions and should have at least five nucleotides complementary to the sequence of interest as described in Molecular Cloning: A Laboratory Manual, 2^{nd} ed., Sambrook, Fritsch and Maniatis, Cold Spring Harbor Laboratory, Cold Spring Harbor, N.Y., 1989.

If used as primers, the composition preferably includes at least two nucleic acid molecules which hybridize to different regions of the target molecule so as to amplify a desired region. Depending on the length of the probe or primer, the target region can range between 70% complementary bases and full complementarity and still hybridize under stringent conditions. For example, for the purpose of detecting the presence of SLE virus and dengue virus, the degree of complementarity between the hybridizing nucleic acid (probe or primer) and the sequence to which it hybridizes is at least enough to distinguish hybridization with a nucleic acid from other organisms.

The nucleic acid sequences encoding SLE virus and dengue virus can be inserted into a vector, such as a plasmid, and recombinantly expressed in a living organism to produce recombinant SLE virus and dengue virus peptide and/or polypeptides.

The nucleic acid sequences in the embodiments described herein also include a diagnostic probe that serves to report the detection of a cDNA amplicon amplified from the viral genomic RNA template by using a reverse-transcription/polymerase chain reaction (RT-PCR), as well as forward and reverse amplimers that are designed to amplify the cDNA amplicon. In certain instances, one of the amplimers is designed to contain a vaccine virus-specific mutation at the 3′-terminal end of the amplimer, which effectively makes the test even more specific for the vaccine strain because extension of the primer at the target site, and consequently amplification, will occur only if the viral RNA template contains that specific mutation.

Automated PCR-based nucleic acid sequence detection systems have been recently developed. TaqMan assay (Applied Biosystems) is widely used. A more recently developed strategy for diagnostic genetic testing makes use of molecular beacons (Tyagi and Kramer 1996 Nature Biotechnology 14:303-308). Molecular beacon assays employ quencher and reporter dyes that differ from those used in the TaqMan assay. These and other detection systems may used by one skilled in the art.

More embodiments concern the use of one or more of the chimeric viruses described herein or nucleic acids encoding said viruses described herein (e.g., SEQ. ID. NOS.: 5, 6, 7, 8, 9, 10, 11, 12, 13, or 14, including ATCC deposits ______) in immunogenic compositions and/or vaccines or as neutralization antigens. That is, aspects of the invention concern the use of one or more of chimeric viruses described herein or nucleic acids encoding said viruses described herein (e.g., SEQ. ID. NOS.: 5, 6, 7, 8, 9, 10, 11, 12, 13, or 14, including ATCC deposits ______) to inoculate an animal so as to generate neutralizing antibodies. These antibodies can be isolated and sequenced by techniques well known in the art. Additionally, neutralizing monoclonal antibodies can be generated from the inoculated animals using conventional techniques. Accordingly, aspects of the invention concern methods of generating neutralizing antibodies, wherein an animal is provided one or more of the chimeric viruses described herein or nucleic acids encoding said viruses described herein (e.g., SEQ. ID. NOS.: 5, 6, 7, 8, 9, 10, 11, 12, 13, or 14, including ATCC deposits ______) so as to generate neutralizing antibodies, and said polyclonal neutralizing antibodies are purified. Optionally, splenocytes from said animal are obtained and fused to myeloma cells so as to create a hybridoma. Hybridoma clones are created, analyzed, and used to generate monoclonal antibodies that neutralize SLE infection.

Additional embodiments concern the use of one or more of the chimeric viruses described herein to screen and identify compounds that effective at ameliorating or ablating SLE infection. By some approaches, cells harboring one or more of the chimeric viruses described herein are contacted with a candidate agent that ameliorates, reduces, or ablates SLE infection, and the amelioration, reduction, or ablation of the presence of the chimeric virus is measured. Agents that reduce, ameliorate, or ablate the presence of the chimeric virus are then identified. Greater detail on the materials and methods used in the experiments provided herein is provided in the following example.

### EXAMPLE 1

Some of the materials and methods used in the experiments that follow are provided in this example.

Cells and Viruses

Vero cells (African green monkey kidney) were maintained in OptiPro SFM (Invitrogen, Grand Island, N.Y.) supplemented with 4 mM L-glutamine (Invitrogen). SH-SY5Y cells (human neuroblastoma) were maintained in D-MEM/F-12 (Invitrogen) supplemented with 10% fetal bovine serum (FBS), 1 mM L-glutamine, and 0.05 mg/ml gentamicin (Invitrogen). C6/36 cells (Aedes albopictus mosquito cells) were maintained at 32° C. in Minimal Essential Medium containing Earle's salts and 25 mM HEPES buffer (Invitrogen) and supplemented with 10% FBS, 2 mM L-glutamine, and 0.1 mM non-essential amino acids (Invitrogen).

A mouse-brain-derived suspension of the SLE Hubbard strain was obtained from the World Reference Center for Emerging Viruses and Arboviruses at the University of Texas Medical Branch, Galveston, Tex. The SLE Hubbard strain was originally isolated from the brain of a deceased patient in Missouri in 1937. For the present study, a virus stock was prepared in Vero cells that had a titer of 10⁶ PFU/ml and is referred to here as uncloned SLE. Subsequently, the virus was biologically-cloned by two successive passages at terminal end-point dilution and finally amplified in Vero cells. This biologically-cloned SLE stock was used as a parental wild-type virus for animal studies and as a source of genomic RNA to prepare the cDNA of prM and E genes for chimeric virus constructions. Complete genome sequence analysis of uncloned SLE (GenBank accession no. EU566860) and the biologically-cloned SLE revealed two putative Vero cell-adaptation mutations: an Arg₂₃₆→Lys substitution located in the E protein and a Met₁₅₃→Val substitution in the NS4B nonstructural protein.

Construction and Recovery of Antigenic Chimeric SLE/DEN4 Viruses

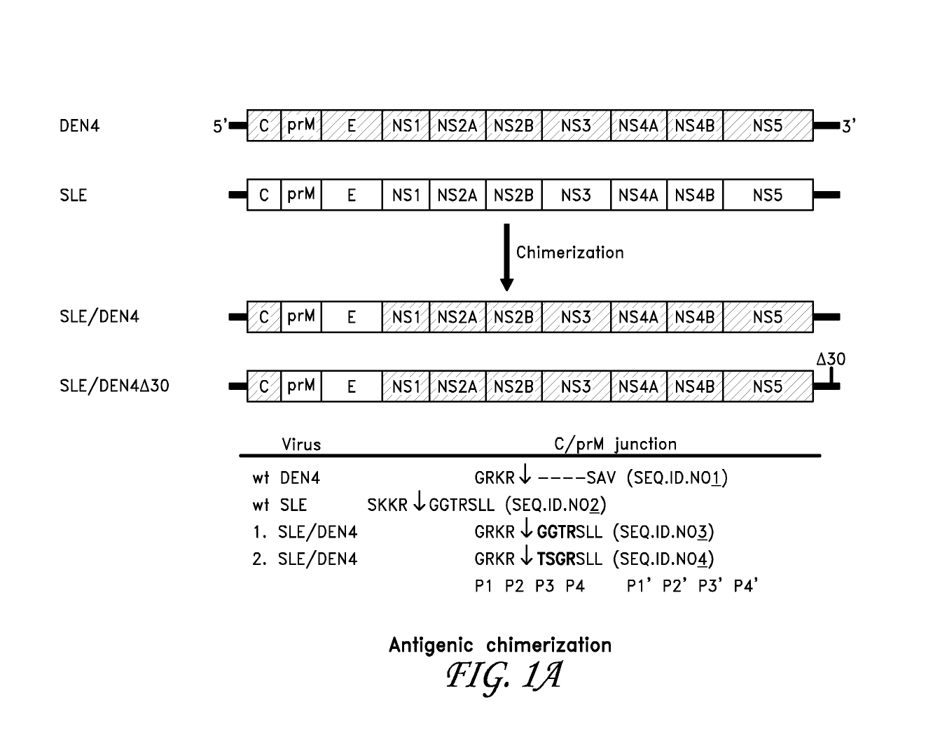

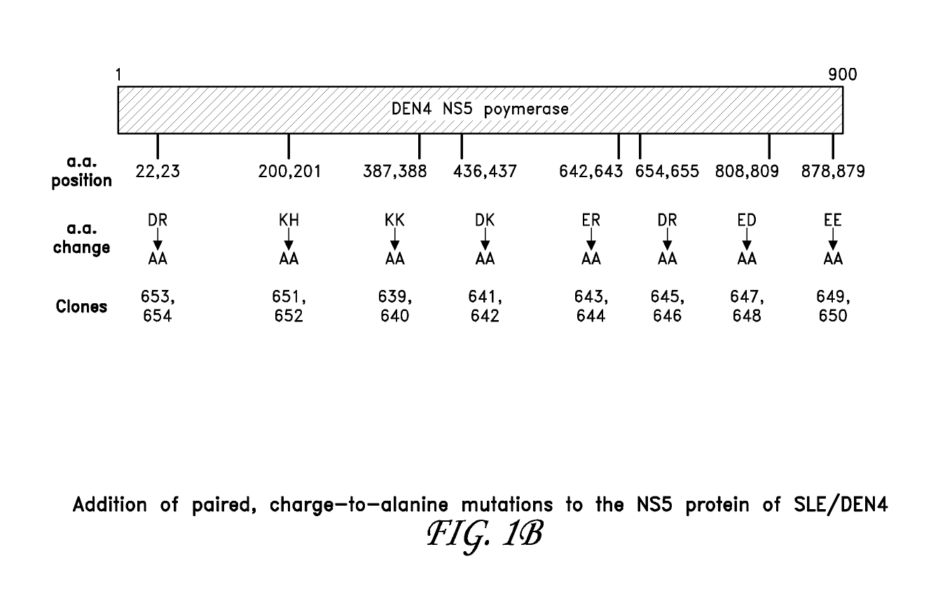

The cDNA clones used to derive the SLE/DEN4 and SLE/DEN4Δ30 antigenic chimeric viruses were generated in a similar manner as was previously performed for the WN/DEN4 chimeric viruses (FIG. 1A) [8]. The p4 plasmid, that contains the full-length infectious cDNA for recombinant DEN4 (rDEN4), was used for the construction of chimeric SLE/DEN4. The source of the SLE cDNA was a PCR product that included nucleotides 408 to 2514 of the SLE genome. Two nucleotide (nt) changes (A>G at nt 1670 and T>C at nt 1700) were identified in the PCR fragment coding for the E protein, neither of which resulted in amino acid substitutions.

For construction of the SLE/DEN4 cDNA plasmid, a PCR fragment containing the SP6 promoter and the 5′ UTR and C gene of DEN4 was generated using forward primer 5′-TGACCATTTCCGGGCGCGCCACGGCGTTAC-3′ (SEQ. ID. NO.: 15) and reverse primer 5′-CAATGTTATACTAGTCCTTITTCTCCCGTTCAA-3′ (SEQ. ID. NO.: 16) and was ligated into a modified polylinker region in pBR322. The forward primer contains an AscI restriction site while the reverse primer contains a SpeI restriction site. The addition of the SpeI site altered the C/prM junction region in SLE by changing two amino acids in capsid, Gly₁₀₄→Thr and Gly₁₀₅→Ser. A PCR fragment containing the prM/E region (nt 416-2386) of SLE was generated using a forward primer (5′-AGAAAAAGGACTAGTGGCAGATCGTTGCTC-3′) (SEQ. ID. NO.: 17) that contains a SpeI restriction site sequence and a reverse primer (5′-GAGTCAGCGAGATGCTCCTGTCGCTCGAGTGCAACCCCATC-3′) (SEQ. ID. NO.: 18) that contains a XhoI site. This fragment was inserted into the modified pBR322 construct containing the SP6 promoter and the 5′UTR and C sequences of DEN4. Finally, the 5′UTR/C/prM/E fragment was inserted into p4 and p4Δ30 after digestion with AscI and XhoI to generate SLE/DEN4 and SLE/DEN4Δ30 that contain Thr-Ser-Gly-Arg (TSGR) (SEQ. ID. NO.: 19) at the P1′-P4′ positions of the C/prM proteolytic cleavage junction (FIG. 1A). A second set of SLE/DEN4 and SLE/DEN4Δ30 plasmids was generated by site-directed mutagenesis and contain Gly-Gly-Thr-Arg (GGTR) (SEQ. ID. NO.: 20) at the C/prM junction (FIG. 1A). The correct full-length chimeric virus genomes were confirmed by sequence analysis.

For recovery of viruses, 5′-capped RNA transcripts were synthesized in vitro from cDNA plasmids and transfected into either Vero cells or C6/36 cells. Briefly, plasmids were linearized with Acc65I and transcribed in vitro using SP6 polymerase. Purified transcripts were then transfected into Vero or C6/36 cells using DOTAP liposomes (Roche, Indianapolis, Ind.). Recovered viruses were amplified by passage in Vero cells and biologically-cloned by two or three terminal dilutions in Vero cells before experimental stocks were prepared. Titration of virus stocks was performed using a plaque assay in Vero cells with visualization of plaques by immunostaining with SLE-specific hyperimmune mouse ascitic fluid (ATCC, Manassas, Va.).

Generation of SLE/DEN4 Viruses with Paired Charge-to-Alanine Mutations

Eight paired charge-to-alanine mutations in the DEN4 NS5 gene that were previously described [14] were individually introduced into the SLE/DEN4 cDNA clone (FIG. 1B). Fragments containing the desired paired charge-to-alanine mutation were excised from the previously constructed mutant p4 plasmids by restriction digest and introduced into the SLE/DEN4 cDNA clone containing the GGTR (SEQ. ID. NO.: 20) C/prM junction. Two sister plasmids were generated for each of the eight paired charge-to-alanine mutations for a total of 16 plasmids. Each plasmid was confirmed to have the correct paired charge-to-alanine mutation by sequence analysis. Viruses were recovered in C6/36 cells after transfection as described above and then passaged in Vero cells to reach a minimum virus titer of approximately 10⁶ PFU/ml. Viruses were biologically-cloned by two or three terminal dilutions before experimental stocks were prepared in Vero cells. The resulting virus stocks were subjected to partial genome sequence analysis to confirm that the virus contained the SLE prM/E region and to determine if the correct paired charge-to-alanine mutation was present.

Studies in Mice

All animal study protocols were approved by the NIAID Animal Care and Use Committee. Viruses were analyzed for neuroinvasiveness by intraperitoneal (IP) inoculation of 3-week-old, female immunocompetent Swiss Webster (SW) mice or SCID mice in groups of 5 or 10. SCID mice (ICRSC-M; Taconic, Germantown, N.Y.) were administered ten-fold serial dilutions of virus in a 0.1 ml volume and were monitored daily for 49 days for signs of encephalitis. Moribund mice were humanely euthanized.

Neurovirulence of parental and chimeric viruses was assayed by intracerebral (IC) inoculation of 3-day-old Swiss Webster mice (Taconic). Litters of approximately ten mice were inoculated with a 0.01 ml volume containing serial ten-fold dilutions of virus. Mice were monitored daily for 21 days for signs of encephalitis, and moribund mice were humanely euthanized.

For analysis of virus replication in the mouse brain, 5-day-old Swiss Webster mice were inoculated IC with 10³ PFU of SLE or a chimeric virus. The brains of four mice from each group were removed every other day from day 1 to 21 or until all mice from a group had succumbed to infection. Brains were individually homogenized to give a 10% suspension diluted in phosphate-buffered Hank's balanced salt solution (Invitrogen) supplemented with 7.5% sucrose, 5 mM sodium glutamate, 0.05 mg/ml ciprofloxacin, 0.06 mg/ml clindamycin, and 0.0025 mg/ml amphotericin. Brain suspensions were clarified by low-speed centrifugation and frozen at −80° C. The virus titer in brain suspensions was determined by plaque assay in Vero cells.

Studies in Rhesus Monkeys

Studies in rhesus monkeys were conducted at Bioqual, Inc (Rockville, Md.) following approval of the protocols by the ACUCs of both NIAID and Bioqual, Inc. Groups of rhesus monkeys (Macaca mulatta) were inoculated subcutaneously (SC) with 10⁵ PFU of SLE or an antigenic chimeric virus. In one study, a ten-fold higher dose (10⁶ PFU) of SLE/DEN4Δ30 was administered. Serum was collected for measurement of viremia on days 0-6, 8, and 10 and quantitated by plaque assay in Vero cells. Serum was drawn on day 28 to determine the levels of neutralizing antibody against SLE by plaque reduction neutralization test using wild type SLE (biologically cloned) or SLE/DEN4 as the target virus. Antibody titer was defined as the dilution of serum that neutralized 60% of virus. Neutralizing antibody titers against SLE and SLE/DEN4 were found to be similar, and titers reported in this study were determined using SLE/DEN4. Selected groups of mock and immunized animals were challenged SC with 10⁵ PFU of SLE at day 35. Viremia was determined on days 0-6, 8, and 10 post-challenge. The next example describes in greater detail the preparation and characterization of the SLE/DEN4 and SLE/DEN4Δ30 viruses.

### EXAMPLE 2

This example describes the recovery and sequence analysis of SLE/DEN4 and SLE/DEN4Δ30 viruses. Molecular cloning techniques were used to replace the prM/E region of the rDEN4 and rDEN4Δ30 viruses with the corresponding region of SLE to generate two viruses, SLE/DEN4 and SLE/DEN4Δ30, respectively (FIG. 1A). Previous attempts to generate DEN4 antigenic chimeric viruses with tick-borne encephalitis virus (TBE), Langat, (LGT), and WN indicated that the sequence of the C/prM cleavage junction was important for viability [8, 15, 16]. Therefore, viruses with two different C/prM junctions were generated; GGTR and TSGR which represent amino acids in the P1′-P4′ position of the C/prM cleavage site (FIG. 1A). Cleavage at this site is mediated by the viral NS2B/NS3 protease. The cDNA plasmid clone for each recombinant chimeric construct was designed to include one of two Vero cell adaptation mutations in the DEN4 NS4B gene (Thr₁₀₅→Ile or Leu₁₁₂→Phe) that were previously associated with enhanced replication of DEN4 parental or chimeric viruses in Vera cells [17]. SLE/DEN4 and SLE/DEN4Δ30 viruses with both GGTR and TSGR junctions were successfully recovered in either C6/36 cells or Vero cells followed by adaptation to Vero cell growth by serial passage and terminal dilution. Experimental stocks were then prepared in Vero cells, and each virus achieved titers of greater than 10⁶ PFU/ml, which would permit the economical manufacture of a potential vaccine candidate.

Genomic sequence analysis was performed on each virus stock and the results are summarized in TABLE 1. The number of adventitious mutations that appeared in experimental virus stocks ranged from zero in SLE/DEN4 clone 551 to four mutations in SLE/DEN4 clone 549 and 554. The NS4B Thr₁₀₅→Ile and Leu₁₁₂→Phe mutations indicated in TABLE 1 were introduced into the cDNA clones to enhance recovery and replication in Vero cells, and are therefore not considered adventitious mutations. Two mutations in E (Ile₇₀→Thr and Phe₁₅₆→Ser) appeared in multiple viruses and may be adventitious mutations associated with increased replication in Vero cells.

Previously, a large panel of DEN4 viruses were generated that contained individual paired charge-to-alanine mutations in the NS5 polymerase gene and exhibited reduced replication in mouse brain, suggesting that the mutations might confer reduced neurovirulence [14]. Eight of the previously described paired charge-to-alanine mutations were selected for inclusion in SLE/DEN4 (FIG. 1B). Two virus clones of each modified SLE/DEN4 mutant virus were recovered (total of 16 viruses) in C6/36 cells and propagated in Vero cells. The SLE/DEN4-654,655 virus clones were found to be strongly temperature-sensitive (ts) in Vero cells at 37° C. and were propagated at 32° C., while all other modified virus clones were successfully propagated at 37° C. Sequence analysis of the NS5 region containing the intended paired charge-to-alanine mutations was performed on the final experimental stock of each modified SLE/DEN4 virus. Thirteen of sixteen viruses contained the correct Ala-Ala sequence indicating the presence of the intended paired charge-to-alanine mutation. Three viruses contained sequence that did not match the plasmid sequence from which it was derived. SLE/DEN4-200,201 clone 652 contained an Ala codon at position 200 as designed, but a Val at position 201. SLE/DEN4-808,809 clone 647 contained an Ala codon at position 808, but a Glu at position 809. Finally, SLE/DEN4-808,809 clone 648 contained a Glu codon at position 808, and an Ala at position 809. Despite the presence of unintended coding changes in these three viruses, each of the sixteen modified SLE/DEN4 viruses were evaluated in mice. Results of full-length sequence analysis for two of the sixteen clones that had desirable properties upon subsequent evaluation are included in TABLE 1.

TABLE 1
Mutations identified after Vero cell passage of SLE/DEN4 viruses

| | SEQ. | | | | | | |
|---|---|---|---|---|---|---|---|
| | ID. | C/prM | | | Nucleotide | Nucleotide | Amino acid |
| Virus | NO. | junction | Clone | Gene | position | substitution | change^{a} |
| SLE/DEN4 | 20 | GGTR | 549 | E | 1162 | U→C | Ile₇₀→Thr |
| | | | | E | 1420 | U→C | Phe₁₅₆→Ser |
| | | | | E | 1506 | A→G | Thr₁₈₅→Ala |
| | | | | NS4B | 7196^{b} | A→C | Leu₁₁₂→Phe |
| | | | | 3′ | 10341 | U→C | — |
| | | | | UTR | | | |
| SLE/DEN4 | 20 | GGTR | 551 | NS4B | 7196^{b} | A→C | Leu₁₁₂→Phe |
| SLE/DEN4 | 19 | TSGR | 554 | E | 1162 | U→C | Ile₇₀→Thr |
| | | | | 25 | | | |
| | | | | E | 1362 | U→C | Tyr₁₃₇→His |
| | | | | E | 1420 | U→C | Phe₁₅₆→Ser |
| | | | | NS3 | 4710 | G→A | Val₅₂→Met |
| | | | | NS4B | 7174^{b} | C→U | Thr₁₀₅→Ile |
| SLE/DEN4Δ30 | 20 | GGTR | 545 | prM | 588 | A→G | Lys₄₆→Glu |
| | | | | NS4B | 7196^{b} | A→C | Leu₁₁₂→Phe |
| SLE/DEN4Δ30 | 20 | GGTR | 546 | E | 1162 | U→C | Ile₇₀→Thr |
| | | | | NS1 | 2827 | U→C | Phe₁₂₄→Ser |
| | | | | NS3 | 5857 | U→C | Leu₄₃₄→Pro |
| | | | | NS4B | 7196^{b} | A→C | Leu₁₁₂→Phe |
| SLE/DEN4Δ30 | 19 | TSGR | 609 | E | 1162 | U→C | Ile₇₀→Thr |
| | | | | NS3 | 4879 | A→C | His₁₀₈→Pro |
| | | | | NS4B | 7174^{b} | C→U | Thr₁₀₅→Ile |
| SLE/DEN4- | 20 | GGTR | 641 | E | 1140 | G→U | Ala₆₃→Ser |
| 436,437 | | | | E | 1420 | U→C | Phe₁₅₆→Ser |
| | | | | NS4B | 7196^{b} | A→C | Leu₁₁₂→Phe |
| SLE/DEN4- | 20 | GGTR | 646 | NS2B | 4367 | A→G | Ile₆₇→Met |
| 654,655 | | | | NS4B | 7196^{b} | A→C | Leu₁₁₂→Phe |

^{a}Numbering indicates amino acid position within protein. Only coding changes and nucleotide substitutions within the UTRs are indicated in this table.
^{b}A Vero cell adaptation mutation that was incorporated into the cDNA clone. The next example describes in greater detail the neuroinvasiveness and neurovirulence of SLE/DEN4 and SLE/DEN4Δ30 viruses in mice.

### EXAMPLE 3

This example describes experiments that were conducted to evaluate the neuroinvasiveness and neurovirulence of SLE/DEN4 and SLE/DEN4Δ30 viruses in mice. First, the two parental SLE preparations, namely, uncloned SLE, which has only one passage in Vero cells, and biologically-cloned SLE were compared in suckling SW mice for neurovirulence following IC inoculation and in adult SW mice for neuroinvasiveness following IP inoculation. In side-by-side comparison of the LD₅₀, both uncloned and cloned SLE was (1) highly virulent for 3-day-old mice with an IC LD₅₀ of 0.2 or 0.7 PFU, respectively, and (2) extremely neuroinvasive for 3-week-old SW mice with an IP LD₅₀ of 32 or 5.6 PFU, respectively. These findings indicate that the differences in the sequences between these two preparations did not affect the highly virulent phenotype of SLE, and biologically-cloned SLE can be used as a reference parental virus for comparative study of neurovirulence and neuroinvasiveness of the newly generated chimeric SLE/DEN4 viruses in mice.

SLE/DEN4 and SLE/DEN4Δ30 were compared with biologically-cloned SLE for neuroinvasiveness and neurovirulence in mice. Neuroinvasiveness was assayed by IP inoculation of highly-sensitive SCID mice followed by daily monitoring for signs of encephalitis and moribundity. SLE was found to be highly neuroinvasive for adult SCID mice with an IP LD₅₀ of 3.2 PFU (Table 2). With the exception of the SLE/DEN4 clone 549, the two other SLE/DEN4 clones and the three SLE/DEN4Δ30 clones were found to be restricted for mouse neuroinvasiveness with at least a 30,000- to 300,000-fold reduction in LD₅₀ when compared to SLE. Infrequent paralysis or death (from 10 to 20%) was observed in the permissive SCID mice inoculated with SLE/DEN4 clone 551 and 554 and SLE/DEN4Δ30 clone 545. In addition, the average survival time of mice, which succumbed to infection, was typically three times longer for chimeric virus-inoculated animals than for animals inoculated with SLE. Since the SLE/DEN4 viruses were so strongly attenuated, it appears that chimerization was responsible in large part for the attenuation and the contribution of the Δ30 mutation was not required for reduced neuroinvasiveness. The presence of a GGTR or TSGR C/prM junction did not appear to influence the level of neuroinvasiveness of the tested viruses. One of the six chimeric viruses tested, SLE/DEN4 clone 549, did not appear to have the high degree of reduced neuroinvasiveness observed for the other chimeric viruses (LD₅₀<10⁴ PFU). However, deceased mice inoculated with SLE/DEN4 clone 549 did have a four-fold increase in average survival time when compared to SLE (data not shown). The reason for the increased neuroinvasiveness of clone 549 relative to the other SLE/DEN4 and SLE/DEN4Δ30 viruses may be due to one or more of the adventitious mutations present in the virus.

Neurovirulence was measured by IC inoculation of SW suckling mice and daily monitoring. Biologically-cloned SLE was found to be highly neurovirulent in suckling mice with an IC LD₅₀ of 0.4 PFU (TABLE 2). Four viruses (SLE/DEN4 clone 551 and SLE/DEN4Δ30 clones 545, 546, and 609) had LD₅₀ values that were only 5- to 10-fold different than that of SLE. Therefore, in contrast to the effect of chimerization on the attenuation of neuroinvasiveness, chimerization of SLE with DEN4 did not substantially reduce neurovirulence and the presence of the Δ30 also had no effect. Interestingly, two SLE/DEN4 viruses, clone 549 and clone 554, had greatly reduced neurovirulence; their LD₅₀ values were at least 250-fold and 2,500-fold higher than observed for SLE, respectively. These two viruses share a common mutation (Phe₁₅₆→Ser) in the E glycoprotein that was not observed in the other four viruses tested, and it is possible that this coding change may confer the reduced neurovirulence (TABLE 1). Again, the presence of the GGTR (SEQ. ID. No.:20) or TSGR (SEQ. ID. No.:19) C/prM junction in the chimeric viruses did not appear to influence neurovirulence. SLE/DEN4 viruses with the GGTR (SEQ. ID. No.:20) sequence were arbitrarily chosen for studies in rhesus monkeys. The next example provides greater detail on the neuroinvasiveness and neurovirulence of SLE/DEN4 viruses bearing charge-to-alanine mutations in mice.

TABLE 2
NEUROINVASIVENESS AND NEUROVIRULENCE OF
SLE/DEN4 VIRUSES IN MICE.

| | | | | Neuroinvasiveness | Neurovirulence |
|---|---|---|---|---|---|
| | SEQ. | | | in adult SCID | in suckling SW |
| | ID. | C/prM | | mice | mice |
| Virus | NO. | junction | Clone | LD₅₀ (PFU)^{a} | LD₅₀ (PFU)^{b} |
| SLE | | — | wt | 3.2 | 0.4 |
| SLE/ | 20 | GGTR | 549 | <10⁴ | >10² |
| DEN4 | | | | | |
| SLE/ | 20 | GGTR | 551 | >10⁶ | 4.4 |
| DEN4 | | | | | |
| SLE/ | 19 | TSGR | 554 | >10⁶ | >10³ |
| DEN4 | | | | | |
| SLE/ | 20 | GGTR | 545 | >10⁵ | 2.2 |
| DEN4Δ30 | | | | | |
| SLE/ | 20 | GGTR | 546 | >10⁵ | 3.0 |
| DEN4Δ30 | | | | | |
| SLE/ | 19 | TSGR | 609 | >10⁵ | 2.9 |
| DEN4Δ30 | | | | | |
| SLE/ | 20 | GGTR | 641 | >10⁴ | >10³ |
| DEN4- | | | | | |
| 436,437 | | | | | |
| SLE/ | 20 | GGTR | 646 | >10⁵ | 428 |
| DEN4- | | | | | |
| 654,655 | | | | | |

^{a}21 day-old SCID mice were inoculated intraperitoneally with serial 10-fold dilutions of indicated virus and then monitored for moribundity for 49 days.
^{b}3 day-old Swiss Webster mice were inoculated intracerebrally with serial 10-fold dilutions of indicated virus and then monitored for moribundity for 21 days.

### EXAMPLE 4

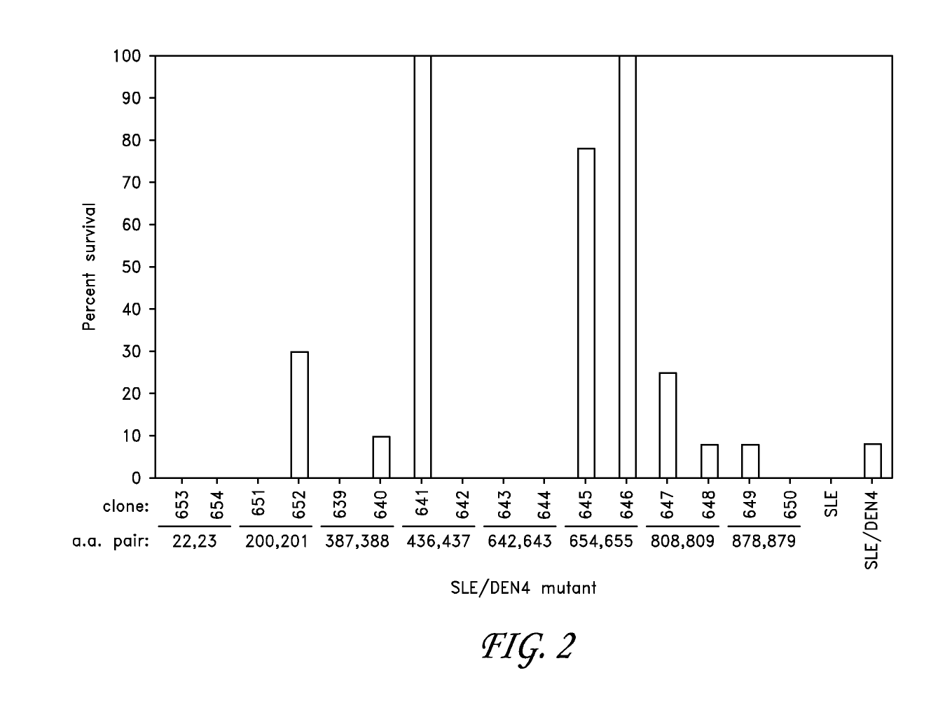

This example describes experiments that were conducted to evaluate the neuroinvasiveness and neurovirulence of SLE/DEN4 viruses bearing charge-to-alanine mutations in mice. Since the SLE/DEN4 viruses retained a high level of neurovirulence for mice, we sought to further attenuate the virus by the introduction of charge-to-alanine mutations that were previously shown to attenuate DEN4 virus for replication in mouse brain (FIG. 1B). An initial experiment was performed to screen the 16 modified SLE/DEN4 viruses for reduced neurovirulence in mice (FIG. 2). Groups of SW suckling mice were inoculated IC with 10² PFU of each virus and compared to biologically-cloned SLE and SLE/DEN4 clone 551. As expected, both SLE and SLE/DEN4 were almost uniformly lethal at this dose. Infection with three of the modified SLE/DEN4 viruses resulted in a substantially greater survival rate. Survival rates were 78% and 100% for mice inoculated with SLE/DEN4-654,655 clone 645 or clone 646, respectively. In the case of SLE/DEN4-436,437, clone 641 was highly attenuated, while all mice inoculated with clone 642 succumbed to infection which suggested that a genetic difference other than the NS5 Asp₄₃₆Lys₄₃₇→AlaAla mutation was likely responsible for the reduced virulence. Complete genomic sequence analysis of clone 641 indicated the presence of an Ala₆₃→Ser substitution and the previously identified Phe₁₅₆→Ser mutation in E, which was also associated with the reduced neurovirulence observed in SLE/DEN4 clones 549 and 554 (TABLE 2). The Phe₁₅₆→Ser mutation in E was not found in any of the other 15 modified SLE/DEN4 viruses. Therefore, based on the association of the presence of the E Phe₁₅₆→Ser mutation with reduced neurovirulence in 3 independent SLE/DEN4 viruses, it is likely that this amino acid change is responsible for the phenotype.

Based on these initial results, SLE/DEN4-436,437 clone 641 and SLE/DEN4-654,655 clone 646 were further evaluated for neuroinvasiveness and neurovirulence by determination of LD₅₀ values in mice (TABLE 2). Like the parental SLE/DEN4 clone 551, the modified viruses had reduced neuroinvasiveness at the highest dose tested in adult SCID mice. In addition, the LD₅₀ values for IC inoculated suckling mice for SLE/DEN4-436,437 clone 641 (>10³ PFU) and SLE/DEN4-654,655 clone 646 (428 PFU) confirmed that these two viruses were significantly attenuated for neurovirulence compared to both SLE and SLE/DEN4.

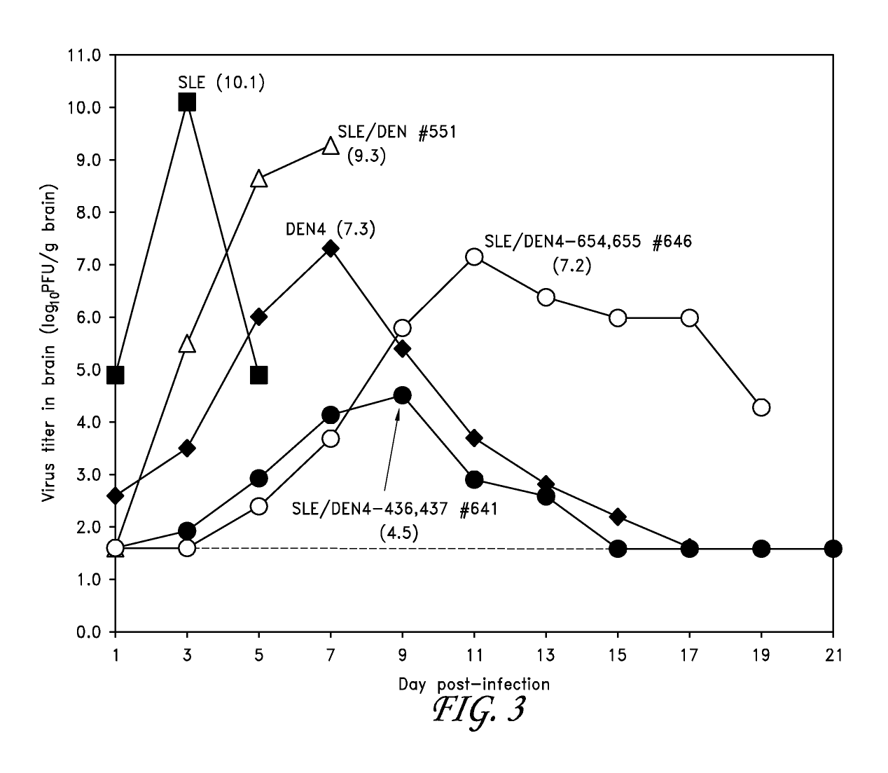

We next sought to quantitate the level of virus replication in mouse brain of SLE, SLE/DEN4, and the further attenuated derivatives, SLE/DEN4-436,437 clone 641 and SLE/DEN4-654,655 clone 646. Similar to previous studies of WN, wild-type SLE rapidly reached an extremely high mean peak virus titer in the brain (10^{10.1} PFU/g) after IC inoculation (FIG. 3) [10]. SLE/DEN4 clone 551 did not appear to be attenuated and reached a mean peak virus titer in the brain of 10^{9.3} PFU/g which was not surprising based on the nearly wild-type level of neurovirulence previously observed (TABLE 2). SLE/DEN4-654,655 clone 646 demonstrated a delay in replication compared to SLE and SLE/DEN4 and reached a mean peak virus titer in the brain of 10^{7.2} PFU/g, an approximately 1,000-fold reduction from SLE indicating a significant level of attenuation for replication in the mouse brain. Even more striking, SLE/DEN4-436,437 clone 641 only reached a mean virus titer of 10^{4.5} PFU/g, which represents a nearly 400,000-fold reduction in replication compared to SLE. The next example describes in greater detail the replication and immunogenicity of the SLE/DEN4 clone 551 and SLE/DEN4Δ30 clone in rhesus monkeys.

### EXAMPLE 5

This example describes experiments that were conducted to evaluate the replication and immunogenicity of the SLE/DEN4 clone 551 and SLE/DEN4Δ30 clone 545 in rhesus monkeys. Two antigenic chimeric viruses, SLE/DEN4 clone 551 and SLE/DEN4Δ30 clone 545, were selected for study in rhesus monkeys because they contained minimal adventitious mutations, which would enable an accurate assessment of the contribution of chimerization and the Δ30 mutation to replication and immunogenicity in rhesus monkeys. SLE/DEN4 clone 551 contains no adventitious mutations, while SLE/DEN4Δ30 clone 545 contains a coding change in prM, Lys₅₄₆→Glu (TABLE 1), which is the only change between the two viruses other than the Δ30 mutation.

Nine of ten monkeys inoculated with biologically-cloned SLE became viremic, and the virus was found to replicate to a mean peak virus titer of 10^{2.1} PFU/ml with a mean number of 3.5 viremic days (TABLE 3). The mean serum neutralizing antibody titer was 1:39 and is lower than reported for other flaviviruses [9, 18-21]. Five of six monkeys inoculated with SLE/DEN4 developed viremia with a mean duration of 2.2 days. The mean peak virus titer (10^{1.1} PFU/ml) was significantly lower than the peak SLE titer (10^{2.1} PFU/ml) (P<0.05). Despite the reduced replication of SLE/DEN4, the mean serum neutralizing antibody titer (1:109) was robust and comparable to antibody levels induced by SLE. In contrast, SLE/DEN4Δ30 was found to be over-attenuated in rhesus monkeys as no monkeys developed detectable viremia, and neutralizing antibodies were not detected. In a separate experiment, four monkeys were inoculated with a ten-fold higher dose of SLE/DEN4Δ30 (10⁶ PFU) and were also found to not develop viremia or sufficient neutralizing antibody levels indicating that the Δ30 mutation confers strong attenuation or reduced infectivity upon SLE/DEN4 in monkeys.

Based on the reduced mouse neurovirulence and restricted replication in mouse brain of SLE/DEN4-436,437 clone 641 and SLE/DEN4-654,655 clone 646, these two viruses were next evaluated for replication and immunogenicity in rhesus monkeys in a comparative study including SLE and SLE/DEN4 clone 551, and the cumulative data is shown in TABLE 3. Three of four monkeys immunized with SLE/DEN4-436,437 clone 641 developed viremia, and the mean number days of viremia (2.0 days) and mean peak virus titer (10^{1.0} PFU/ml) were similar to the levels observed in SLE/DEN4-immunized animals. The mean peak virus titer was significantly lower than that of SLE (P<0.05). The antibody response (1:28) induced by SLE/DEN4-436,437 was also comparable to that observed for animals immunized with SLE (1:39) or SLE/DEN4 (1:109). These results indicate that the addition of either the NS5 Asp₄₃₆Lys₄₃₇→AlaAla mutation or the E Phe₁₅₆→Ser mutation in SLE/DEN4 does not confer further attenuation in rhesus monkeys beyond the level conferred by antigenic chimerization. In contrast, the monkeys inoculated with SLE/DEN4-654,655 clone 646 had no detectable viremia and no monkey seroconverted to SLE although weak antibody responses were detected. These results indicate that the presence of the NS5 Asp₆₅₄Arg₆₅₅→AlaAla mutation in SLE/DEN4 had a potentially over-attenuating effect in rhesus monkeys similar to that observed by the Δ30 mutation in SLE/DEN4Δ30.

Groups of monkeys inoculated with SLE, SLE/DEN4, or SLE/DEN4Δ30 (10⁵ PFU dose only) were challenged with 10⁵ PFU of SLE on day 35 after immunization. As expected based on the observed neutralizing antibody responses, SLE- and SLE-DEN4-immunized animals were completely protected from the development of viremia. In contrast, three of four monkeys immunized with SLE/DEN4Δ30 developed viremia after challenge with SLE although the mean duration (1.0 day) and mean peak virus titer (10^{1.1} PFU/ml) was lower than in mock-immunized monkeys challenged with SLE.

The groups of four rhesus monkeys inoculated with SLE/DEN4-436,437 and SLE/DEN4-654,655 were also challenged with SLE on day 35 post-infection. Each animal was protected as demonstrated by a lack of detectable viremia. This was not surprising based on the immunogenicity of SLE/DEN4-436,437, but was somewhat unexpected for monkeys immunized with SLE/DEN4-654,655 since the pre-challenge SLE-specific antibody levels were lower. However, in contrast to the SLE/DEN4Δ30-immunized animals, which had no detectable antibody and were not fully protected from SLE challenge, each of the four SLE/DEN4-654,655-immunized animals had at least a detectable neutralizing antibody titer against SLE. The next example describes in greater detail the evaluation of the temperature sensitivity of the SLE/DEN4 viruses.

TABLE 3
REPLICATION AND IMMUNOGENICITY OF PARENTAL SLE AND
CHIMERIC VIRUSES IN RHESUS MONKEYS

| | | | | Mean no. | | | |
|---|---|---|---|---|---|---|---|
| | | | | of days | Mean peak | Geometric mean | |
| | | No. of | % with | with | virus titer ± SE | serum neutralizing | Seroconversion^{d} |
| Virus^{a} | Clone | monkeys | viremia | viremia | (log₁₀PFU/ml)^{b} | antibody titer^{c} | (%) |
| SLE | wt | 10 | 90 | 3.5 | 2.1 ± 0.2 | 39 | 90 |
| SLE/DEN4 | 551 | 6 | 83 | 2.2 | 1.1 ± 0.2 | 109 | 100 |
| SLE/DEN4Δ30 | 545 | 4 | 0 | 0 | <0.7 | <5 | 0 |
| SLE/DEN4-436,437 | 641 | 4 | 75 | 2.0 | 1.0 ± 0.2 | 28 | 75 |
| SLE/DEN4-654,655 | 646 | 4 | 0 | 0 | <0.7 | 11 | 0 |

^{a}Groups of rhesus monkeys were inoculated SC with 10⁵ PFU of indicated virus. Serum was collected on day 0-6, 8, and 10 for viremia assay and day 28 for antibody titer determination.
^{b}Virus titer in serum was determined by plaque assay in Vero cells.
^{c}Plaque reduction (60%) neutralizing antibody titers were determined using SLE/DEN4 as target virus. The reciprocal dilution is reported.
^{d}Seroconversion defined as a 4-fold or greater increase in serum neutralizing antibody level to SLE/DEN4 on day 28.

### EXAMPLE 6

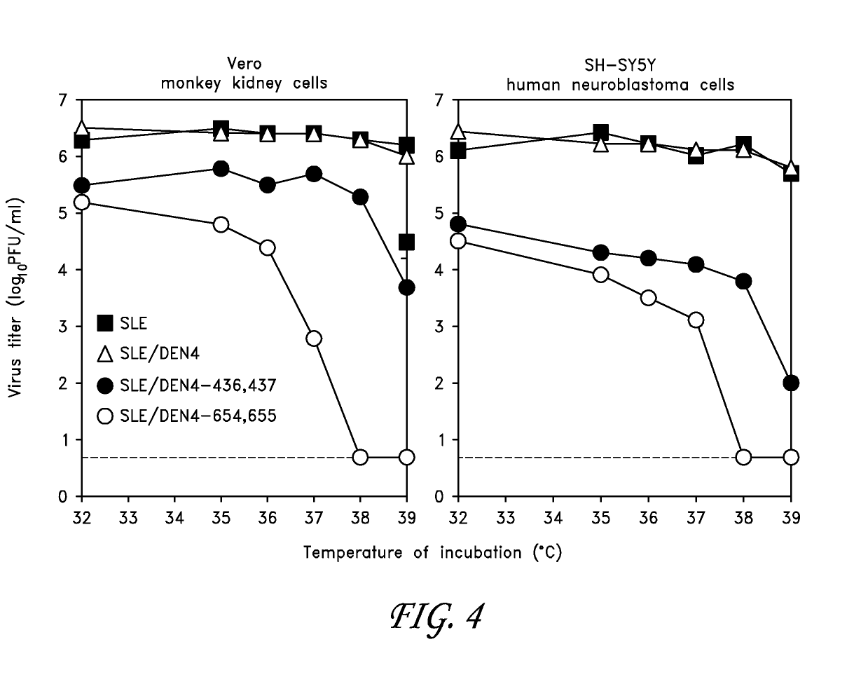

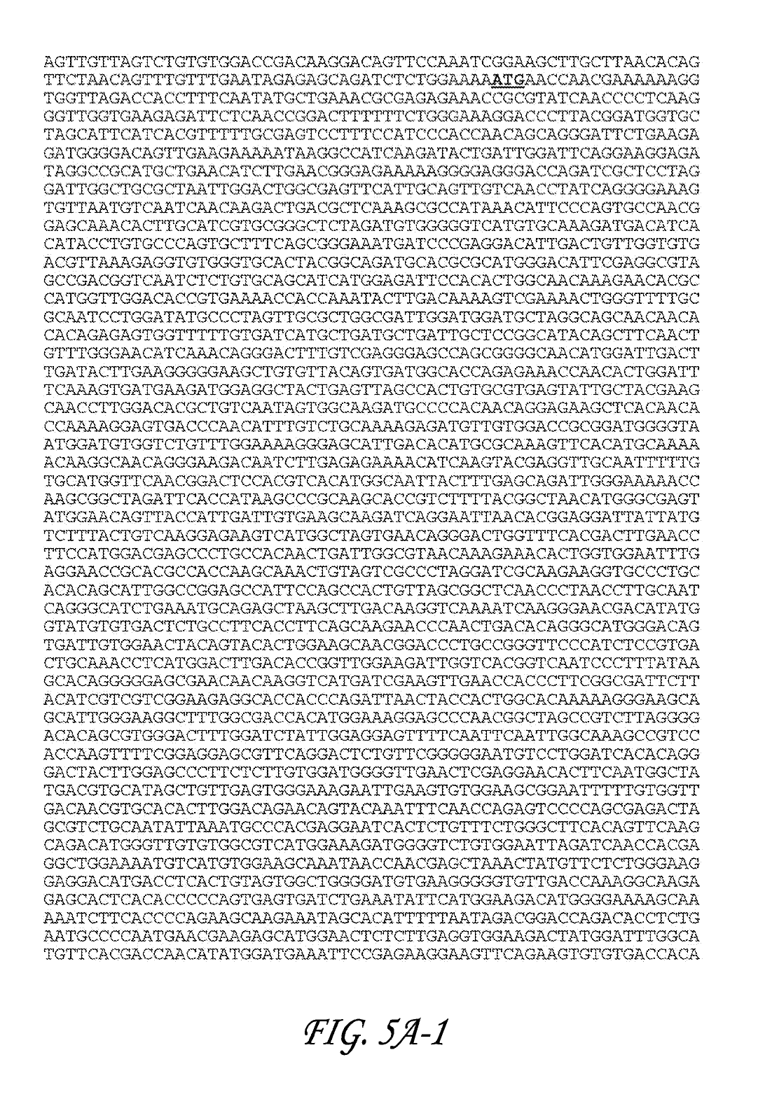

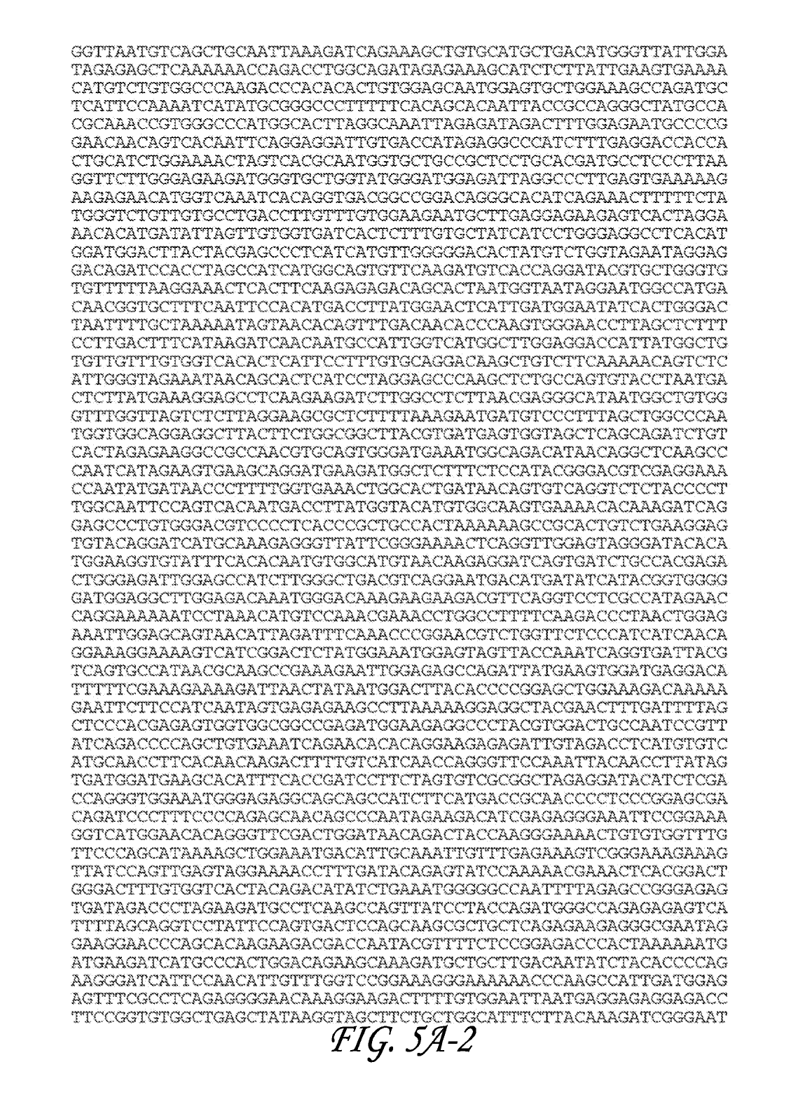

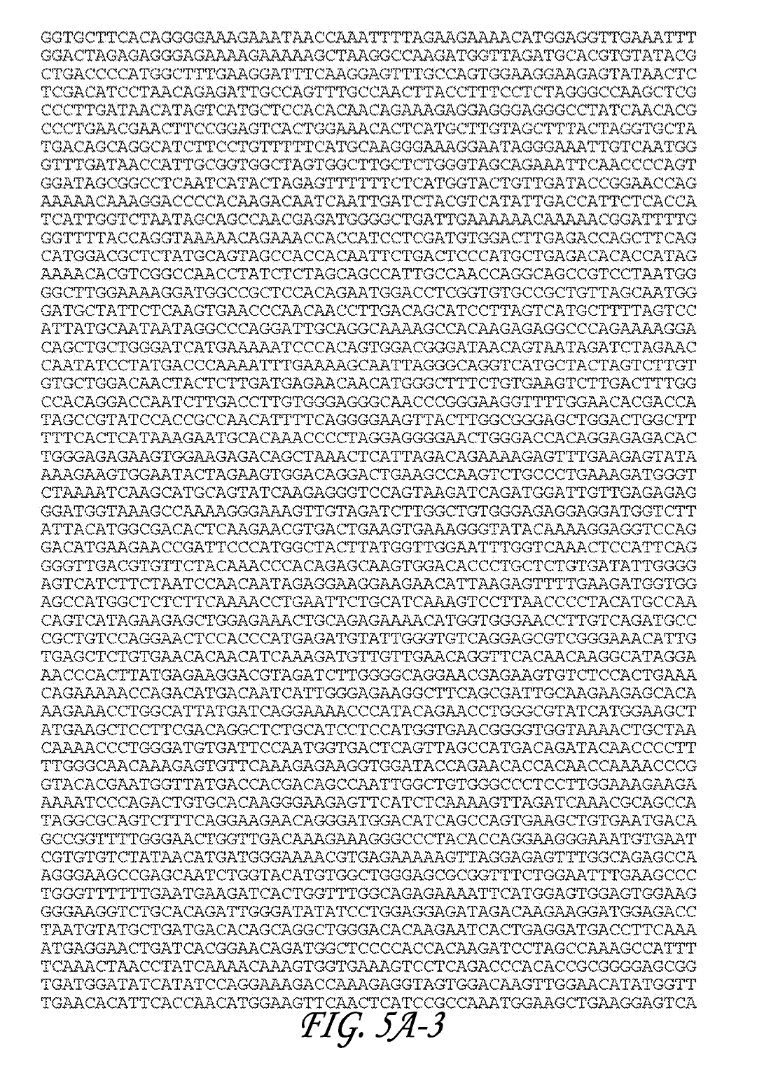

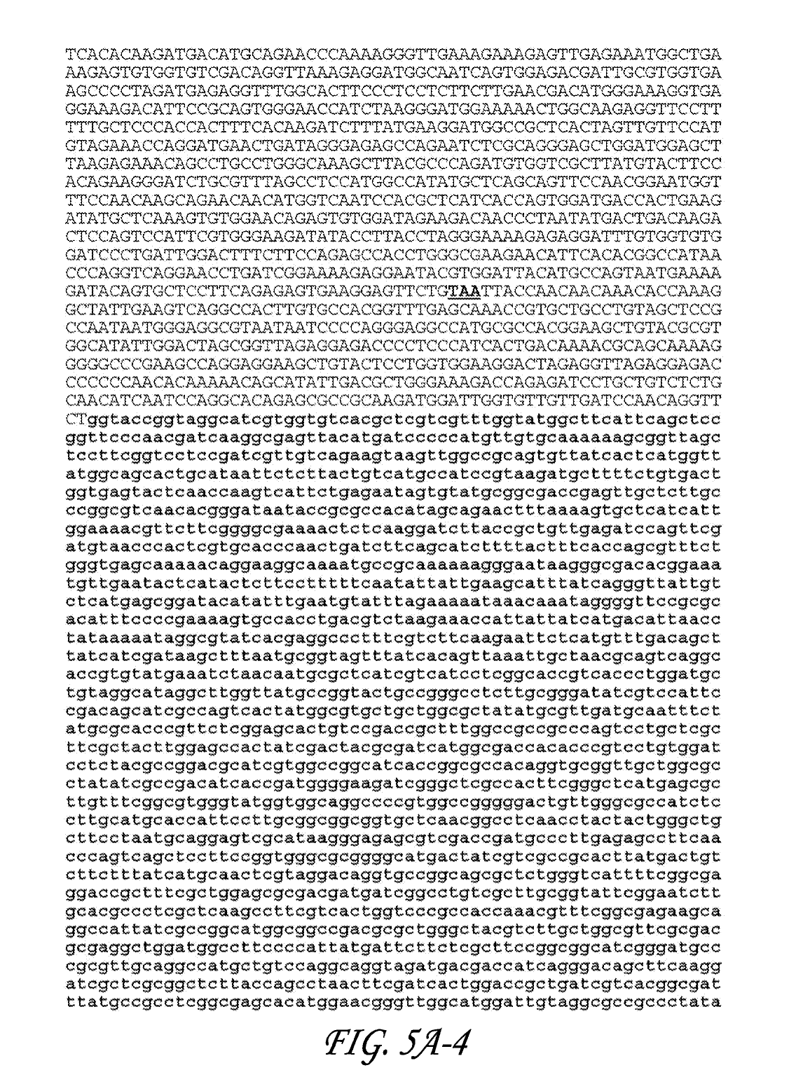

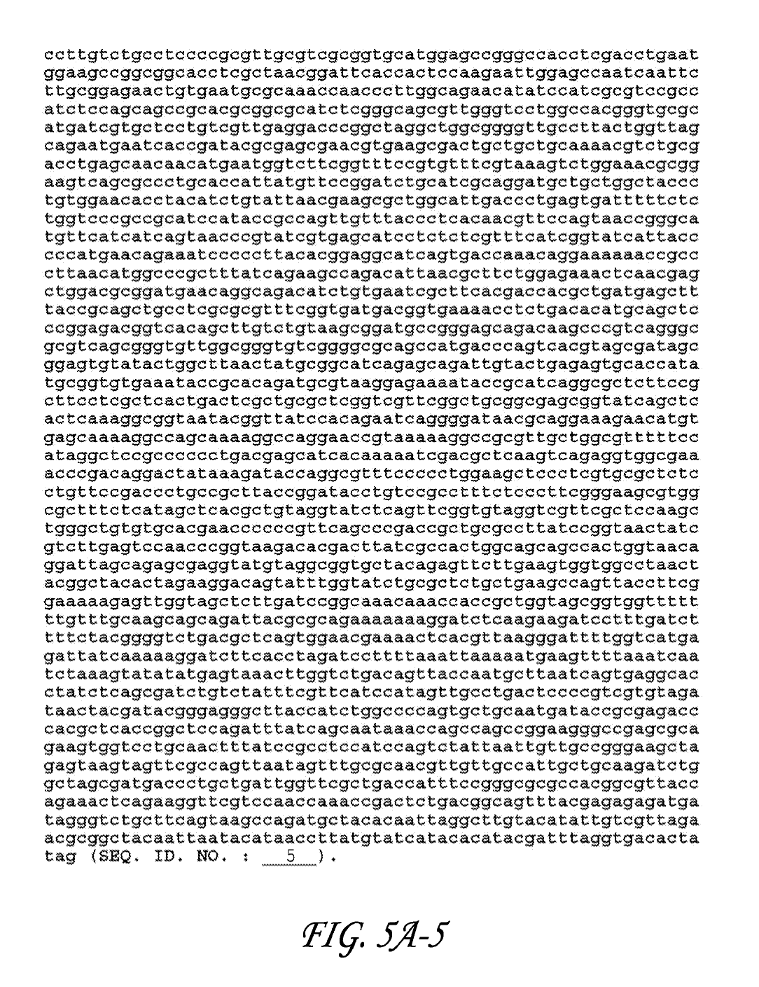

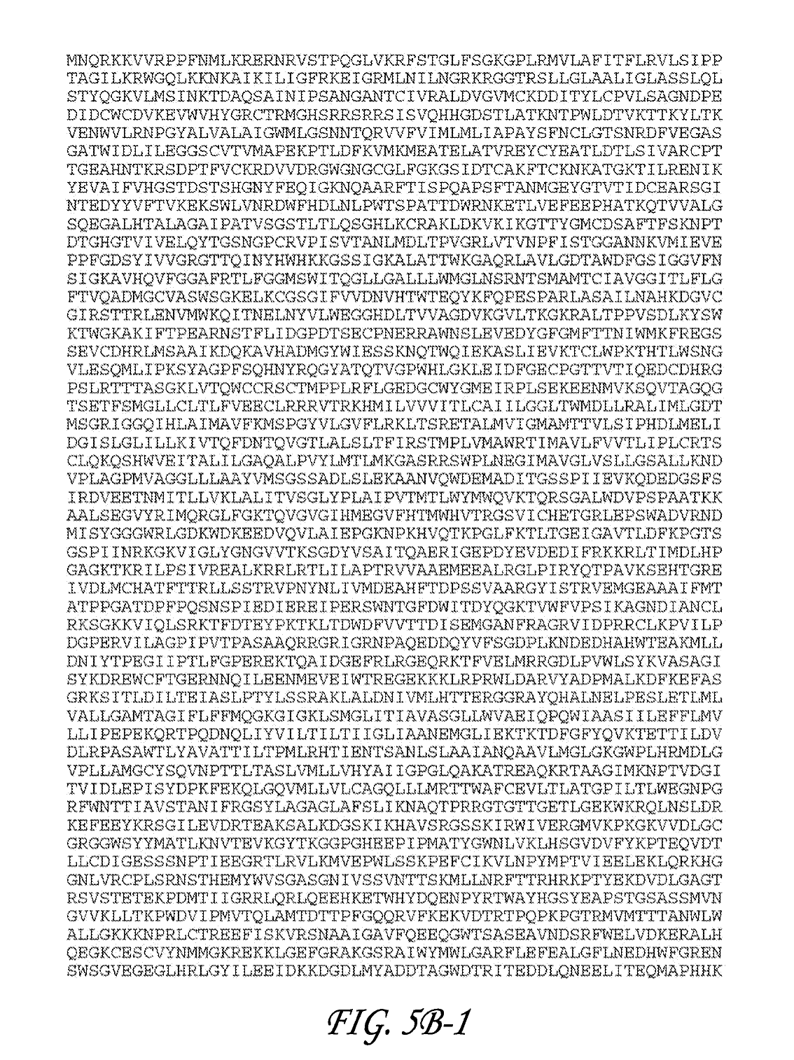

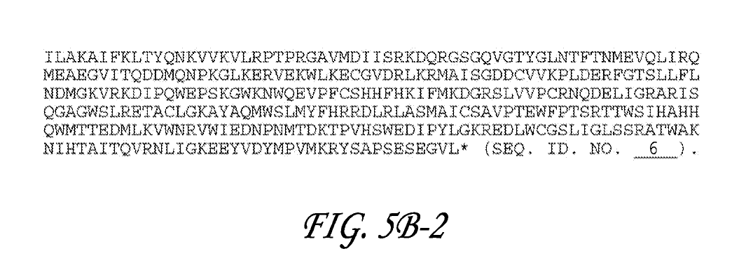

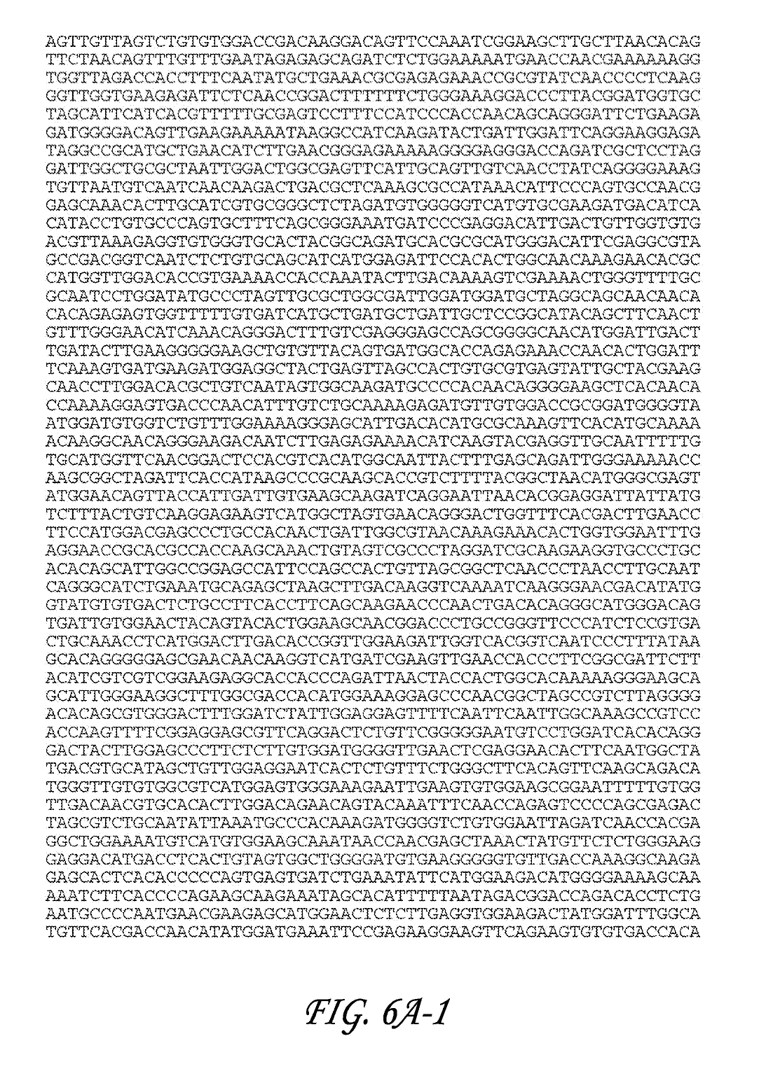

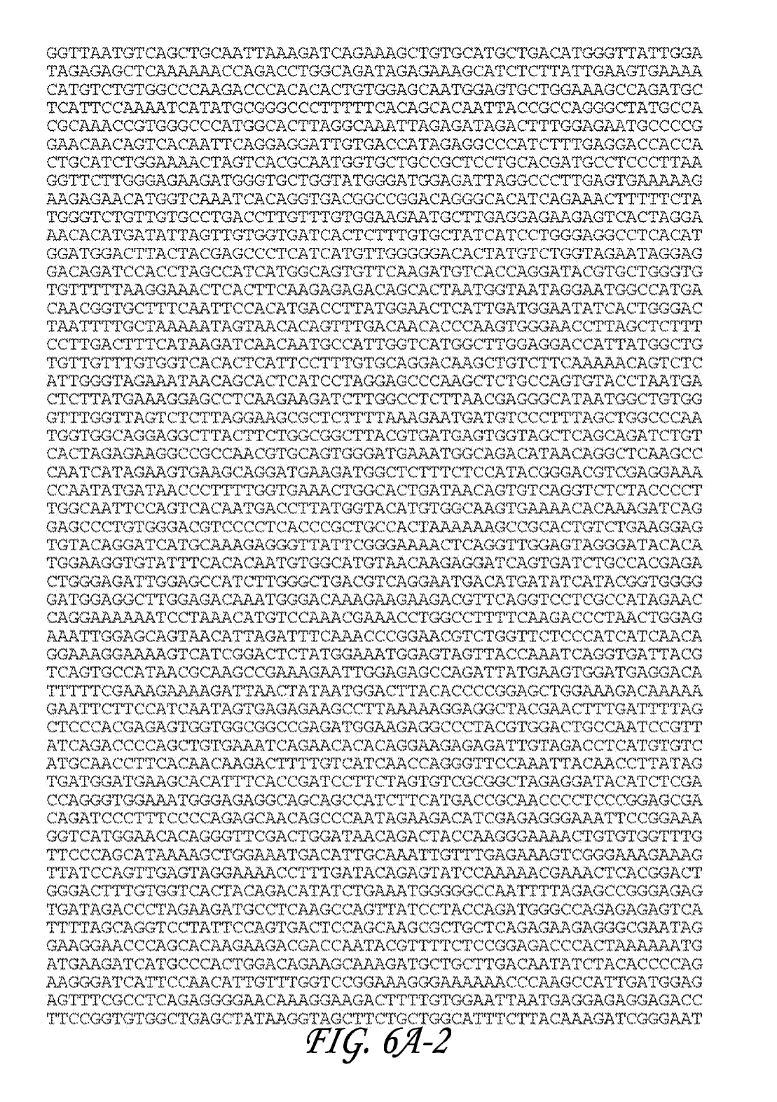

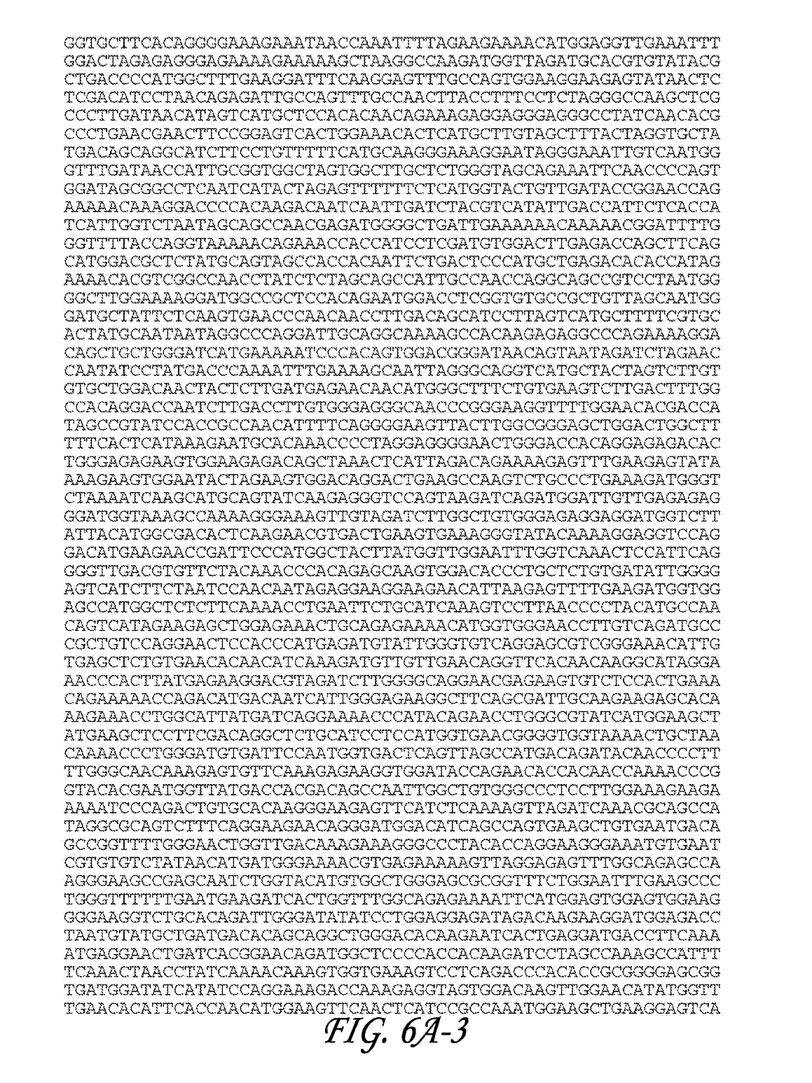

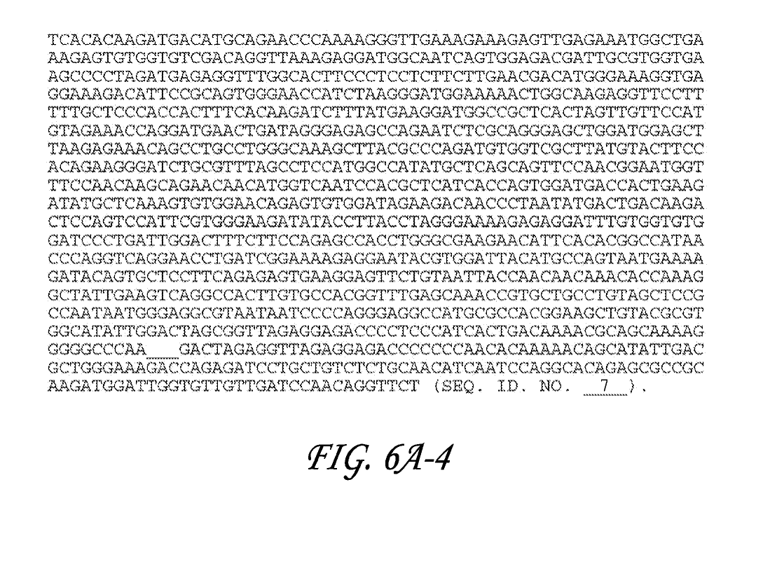

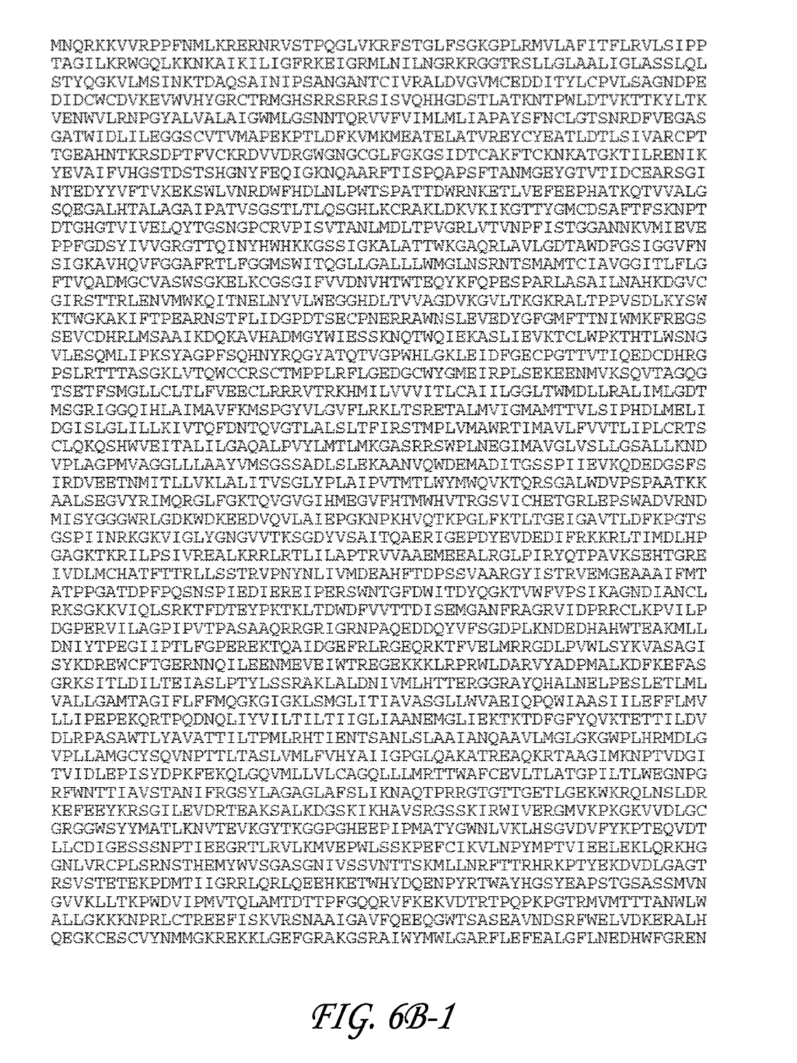

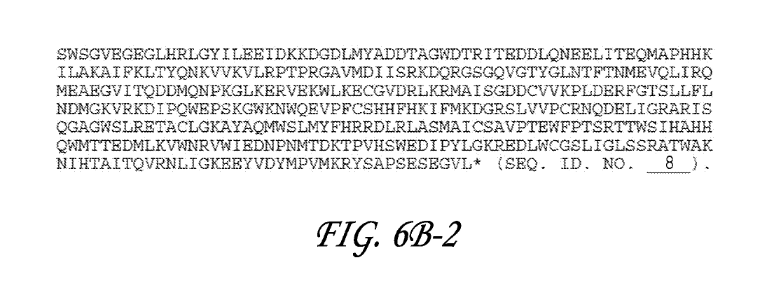

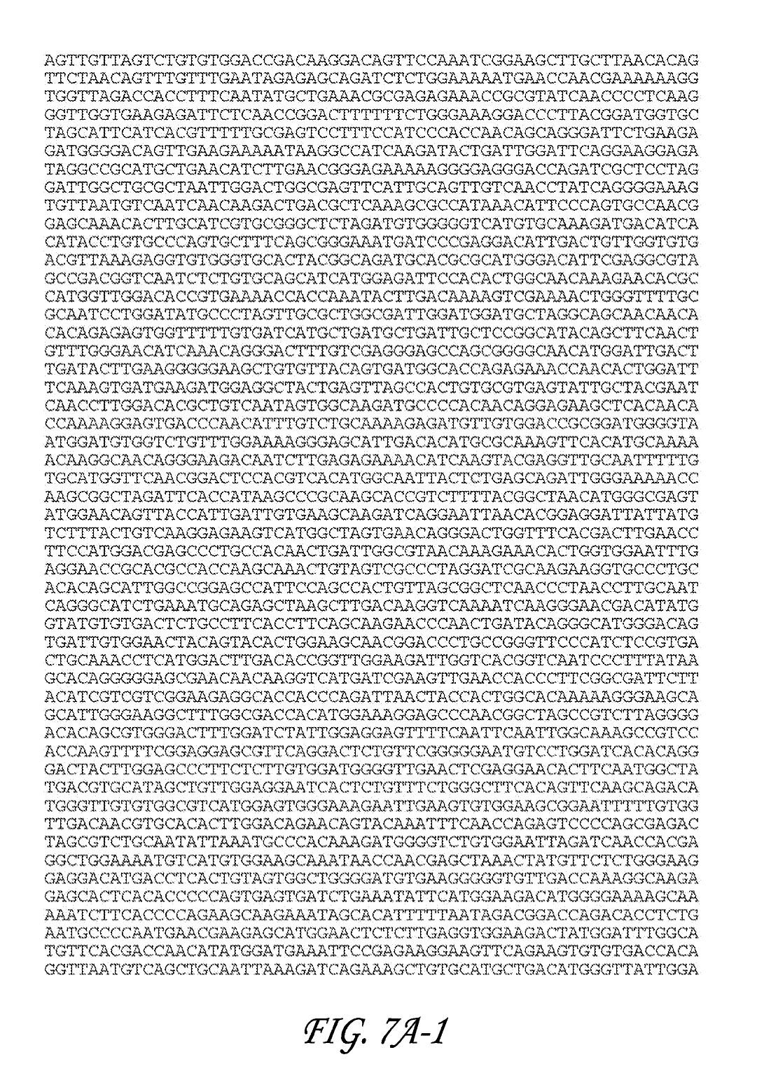

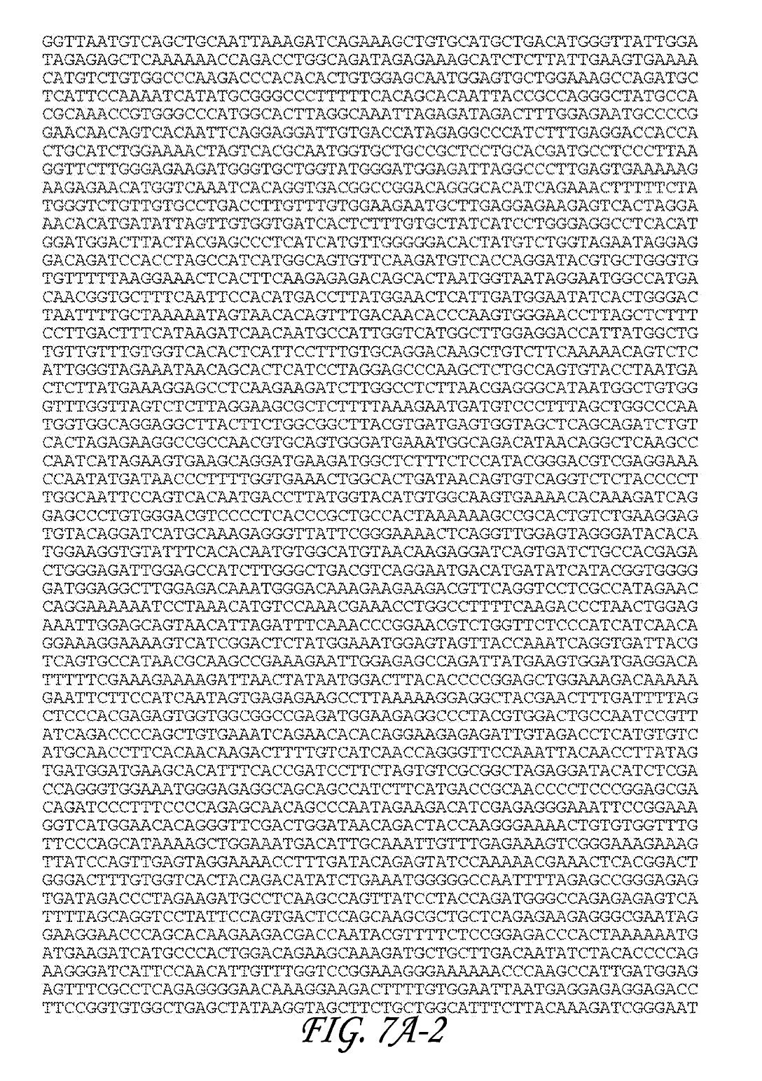

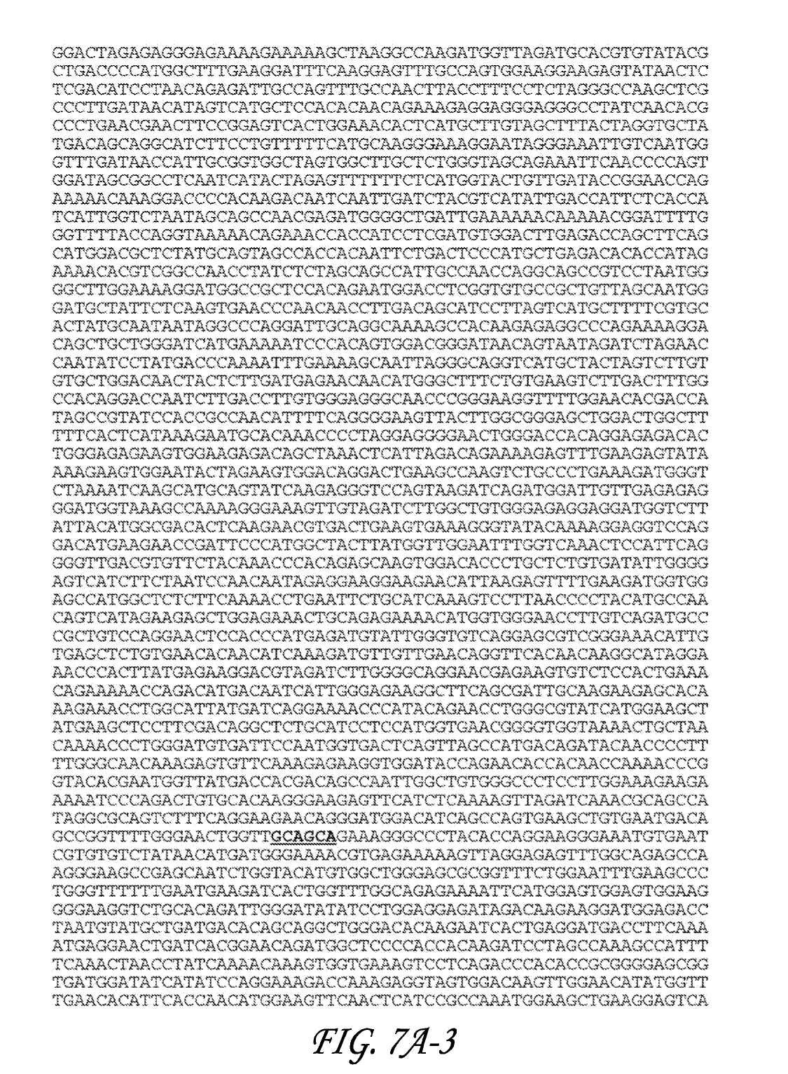

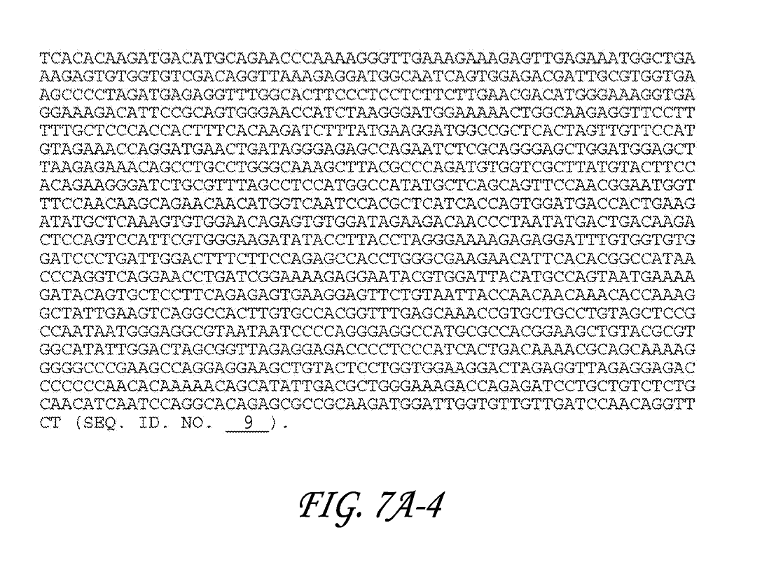

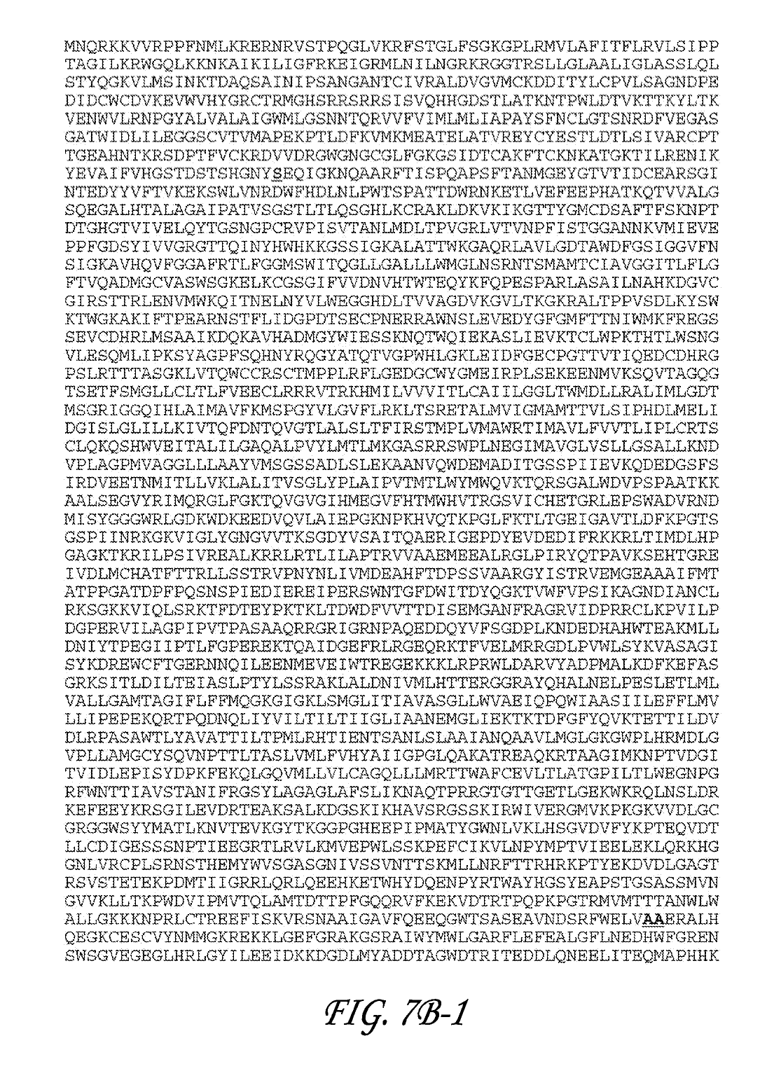

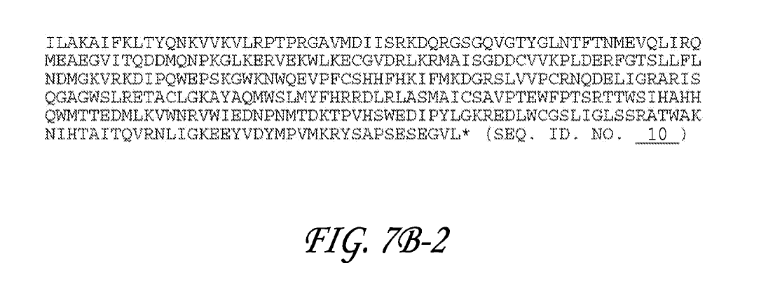

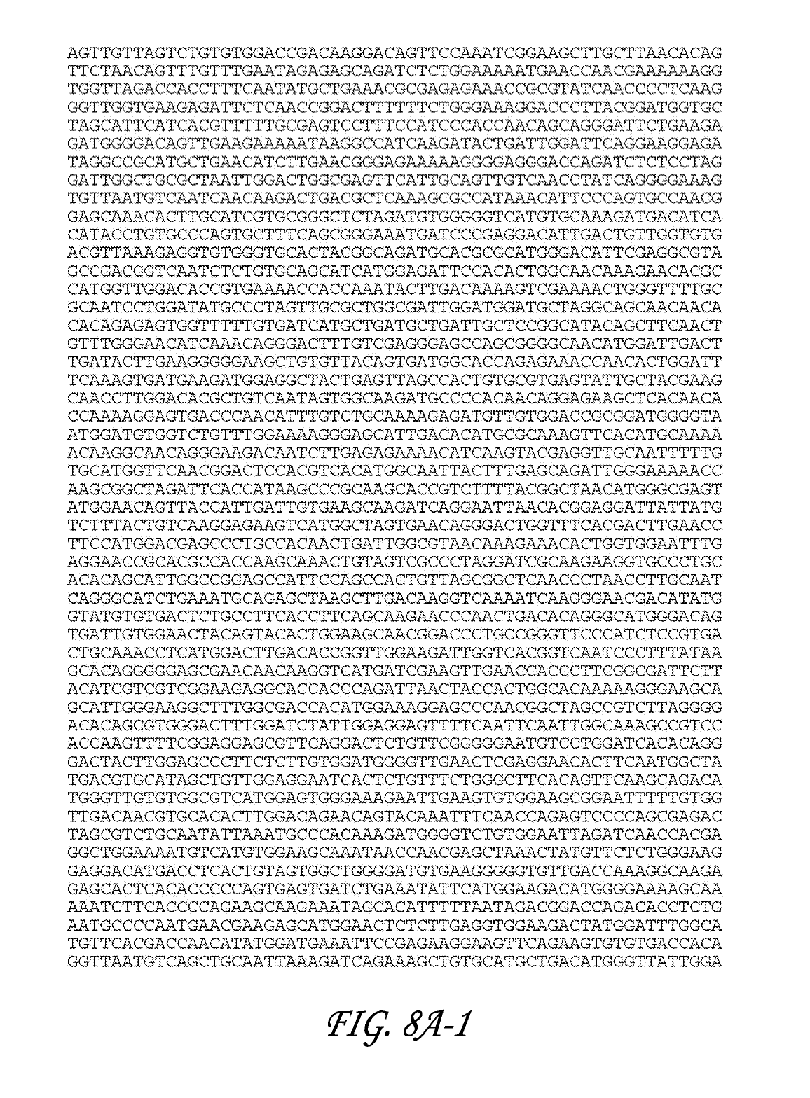

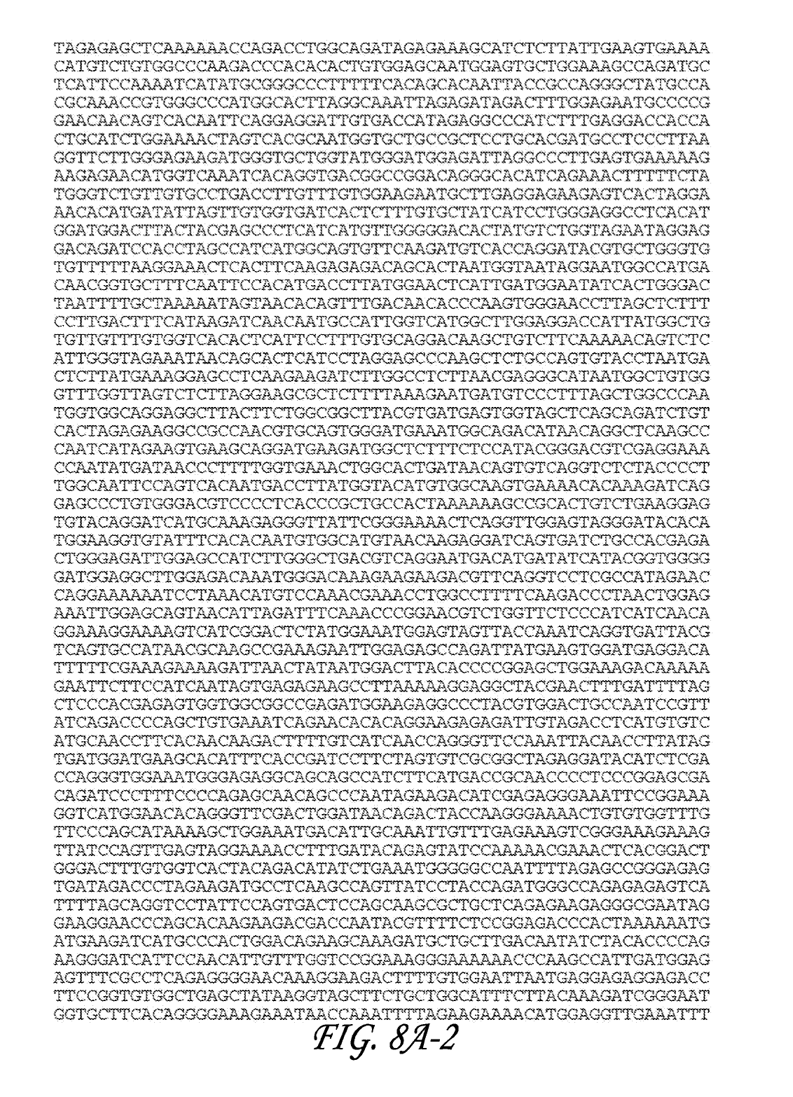

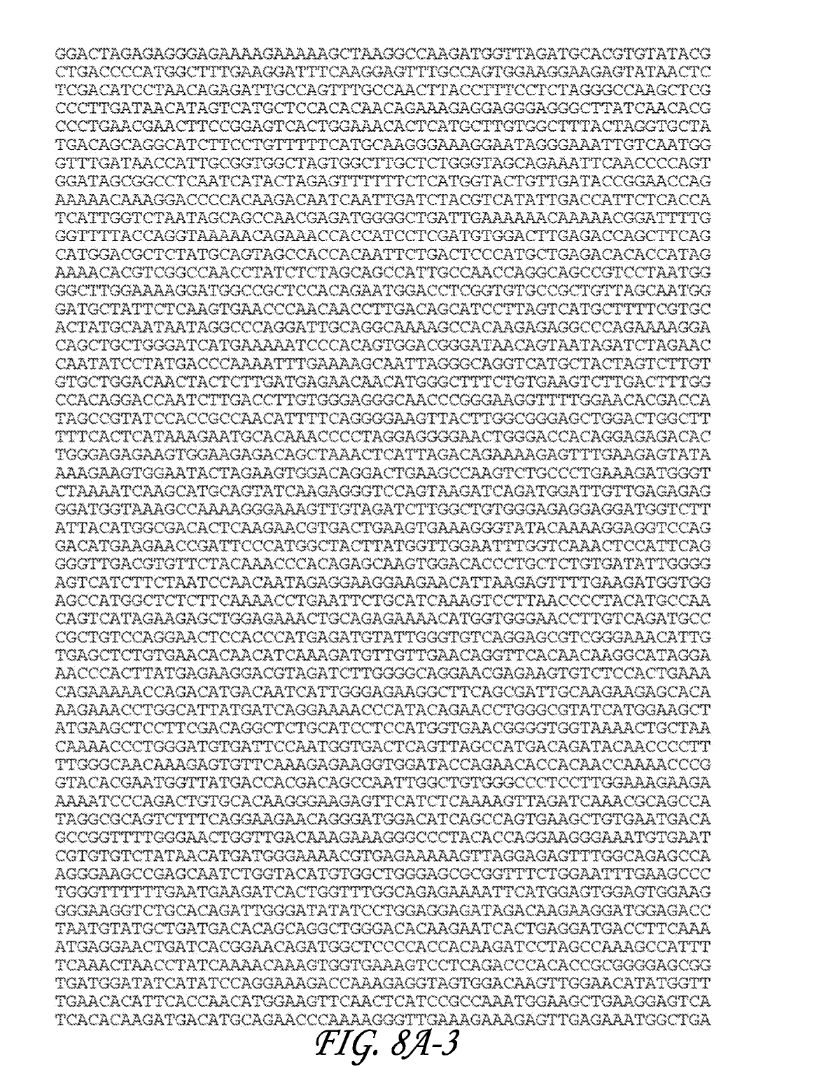

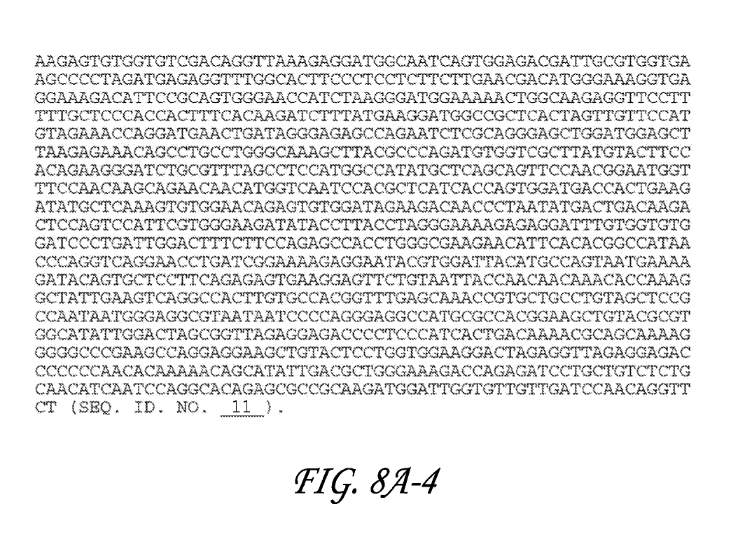

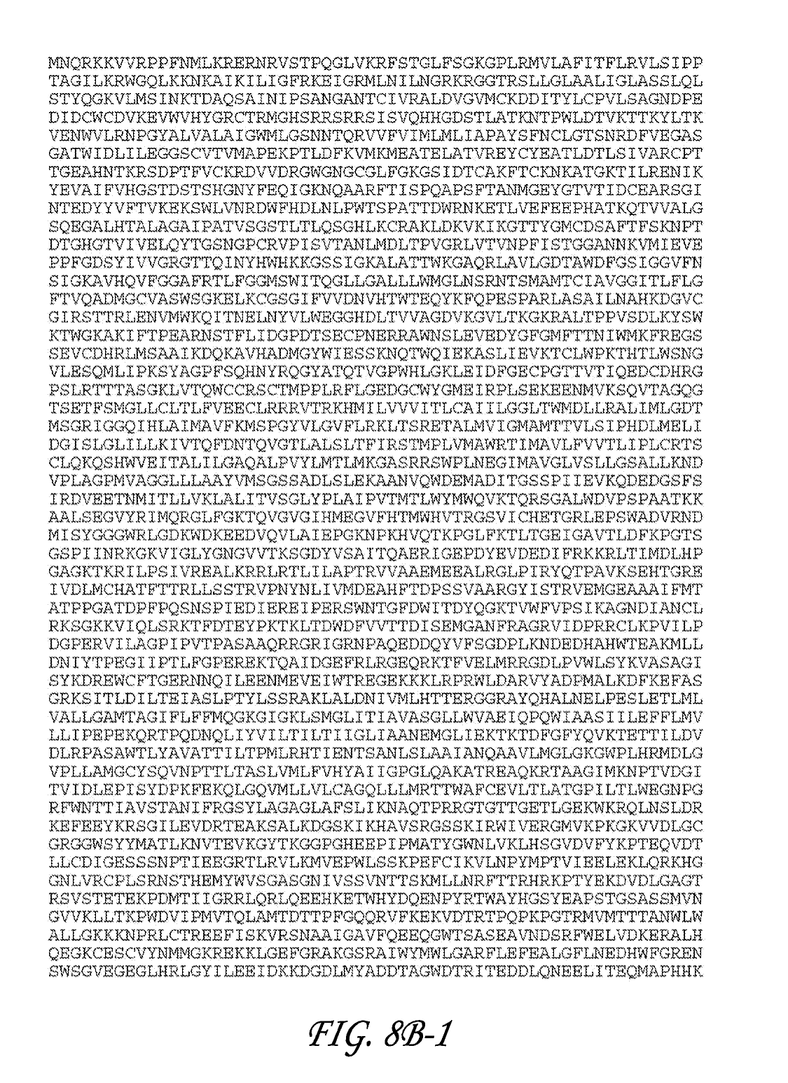

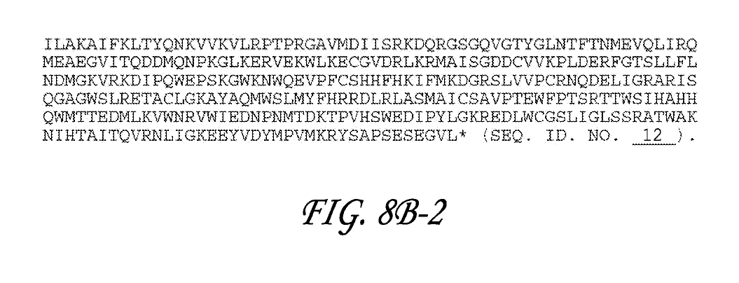

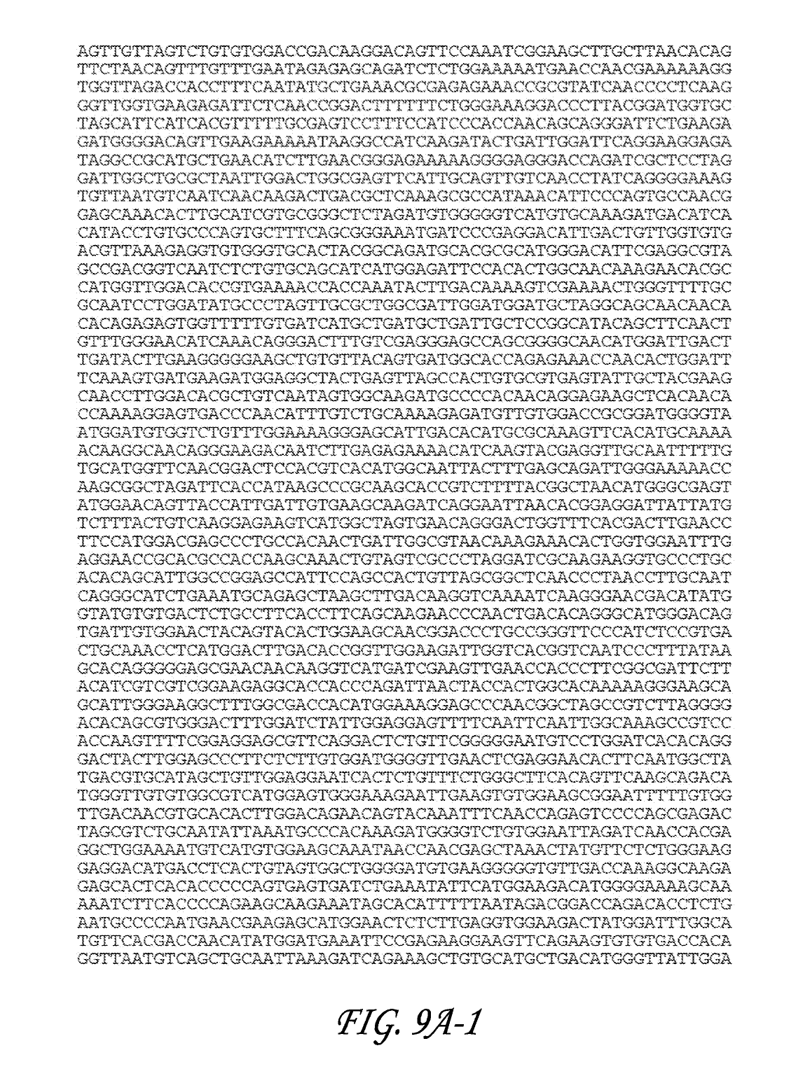

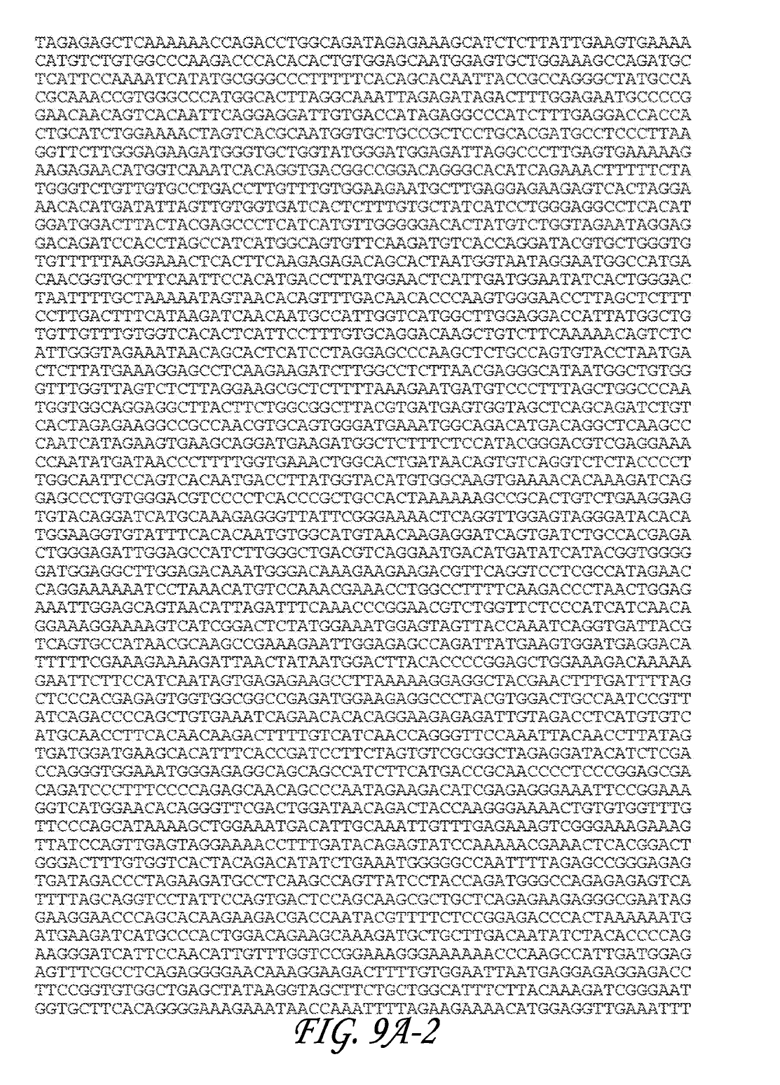

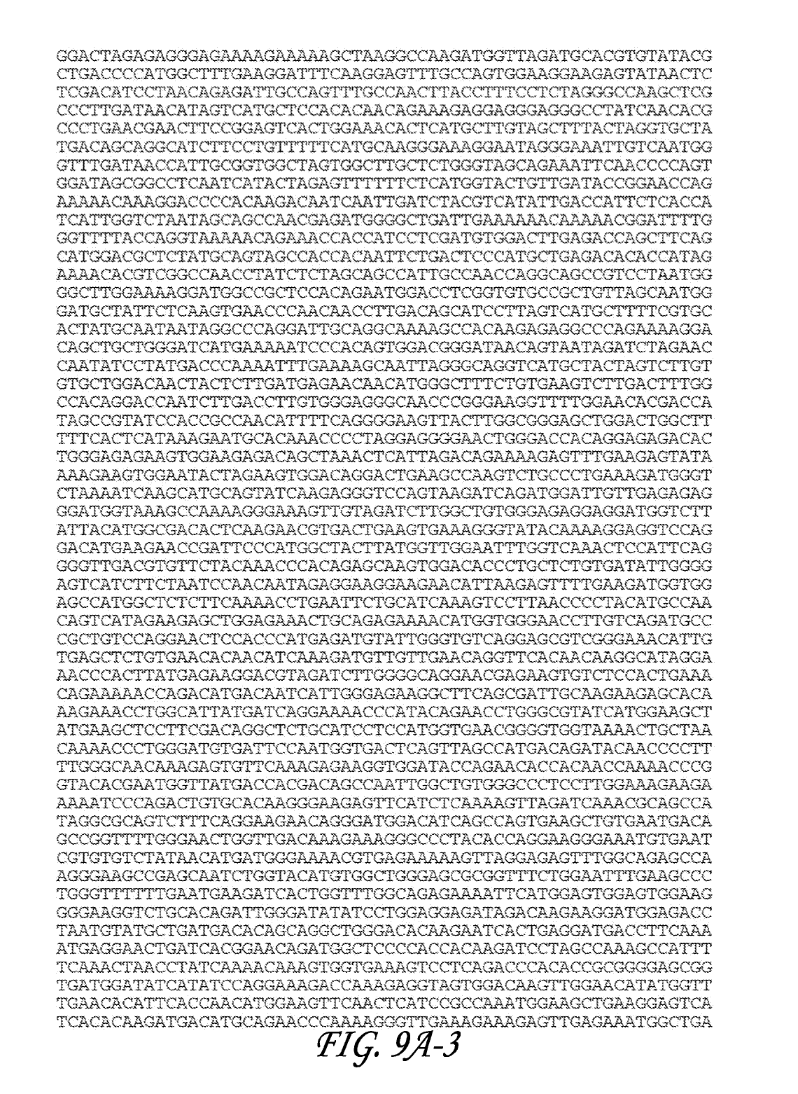

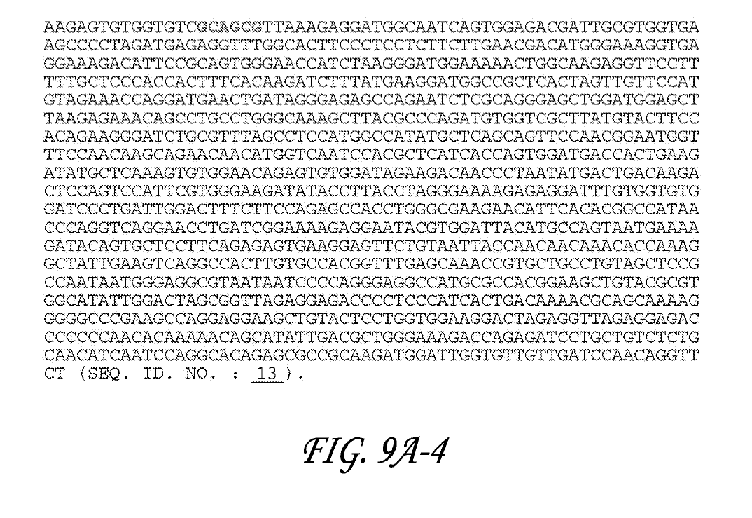

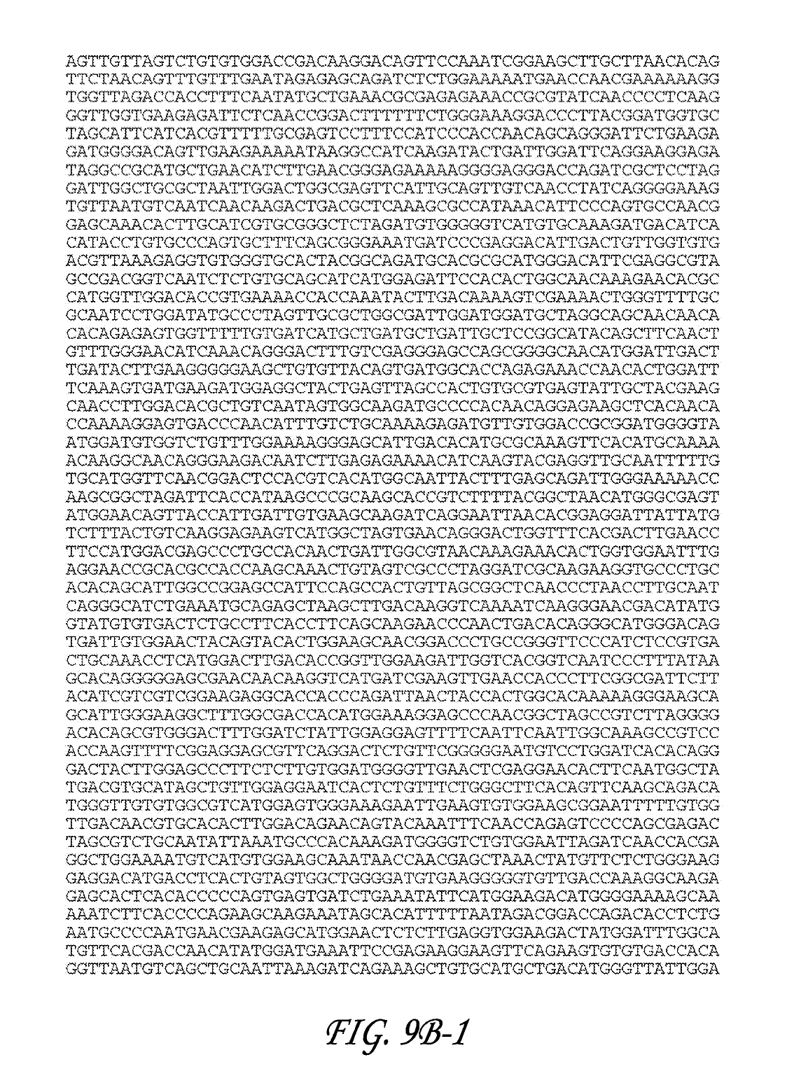

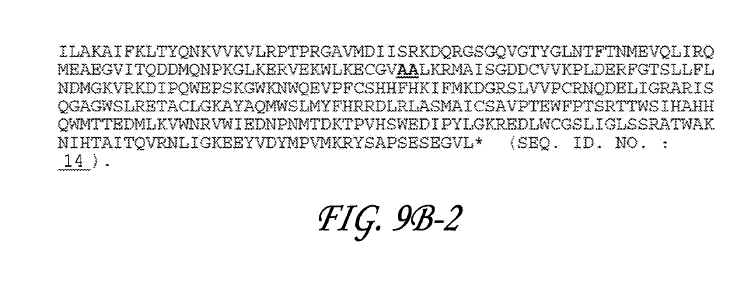

This example describes experiments that were conducted to evaluate the temperature sensitivity of the SLE/DEN4 viruses. To determine if the lack of detectable replication and decreased immunogenicity of SLE/DEN4-654,655 was due to temperature sensitivity of virus replication, the modified SLE/DEN4 viruses were analyzed for plaque formation in Vero cells and SH-SY5Y human neuroblastoma cells at varying temperatures. As mentioned above, SLE/DEN4-654,655 was found to be is upon passage in Vero cells and was propagated at 32° C. SLE and SLE/DEN4 were shown to replicate efficiently at 39° C. (FIG. 4). SLE/DEN4-436,437 clone 641 was found to be moderately ts at 39° C. when compared to permissive temperature, 32° C. In contrast, plaque formation of SLE/DEN4-654,655 was reduced at 37° C. and completely abrogated at 38° C. in both cell types. These results indicate that a strong ts phenotype associated with the NS5 654,655 paired charge-to-alanine mutation may be a factor in the over-attenuation observed in rhesus monkeys. However, temperature sensitivity did not account for the over-attenuation of SLE/DEN4Δ30 in rhesus monkeys, since this virus was not ts at temperatures up to 39° C. The following section lists some of the references that are noted supra.

### REFERENCES

[1] Lindenbach B D, Rice C M. Flaviviridae: the viruses and their replication. In: Knipe D M, Howley P M, editors. Fields Virology. Fourth ed. Philadelphia, Pa.: Lippincott Williams and Wilkins, 2001: 1043-125.
[2] Monath T P. St. Louis encephalitis. Washington D.C.: American Public Health Association, 1980.
[3] Meehan P J, Wells D L, Paul W, Buff E, Lewis A, Muth D, Hopkins R, Karabatsos N, Tsai T F. Epidemiological features of and public health response to a St. Louis encephalitis epidemic in Florida, 1990-1. Epidemiology and infection 2000 August; 125(1):181-8.
[4] Diaz L A, Re V, Almiron W R, Farias A, Vazquez A, Sanchez-Seco M P, Aguilar J, Spinsanti L, Konigheim B, Visintin A, Garcia J, Morales M A, Tenorio A, Contigiani M. Genotype III Saint Louis encephalitis virus outbreak, Argentina, 2005. Emerg Infect Dis 2006 November; 12(11):1752-4.
[5] Mondini A, Cardeal I L, Lazaro E, Nunes S H, Moreira C C, Rahal P, Maia I L, Franco C, Gongora D V, Gongora-Rubio F, Cabrera E M, Figueiredo L T, da Fonseca F G, Bronzoni R V, Chiaravalloti-Neto F, Nogueira M L. Saint Louis encephalitis virus, Brazil. Emerg Infect Dis 2007 January; 13(1):176-8.
[6] Reisen W K. Epidemiology of St. Louis encephalitis virus. Adv Virus Res 2003; 61:139-83.
[7] Burke D S, Monath T P. Flaviviruses. In: Knipe D M, Howley P M, editors. Fields Virology. Fourth ed. Philadelphia: Lippincott Williams and Wilkins, 2001: 1043-125.
[8] Pletnev A G, Putnak R, Speicher J, Wagar E J, Vaughn D W. West Nile virus/dengue type 4 virus chimeras that are reduced in neurovirulence and peripheral virulence without loss of immunogenicity or protective efficacy. Proc Natl Acad Sci USA 2002; 99(5):3036-41.
[9] Pletnev A G, Claire M S, Elkins R, Speicher J, Murphy B R, Chanock R M. Molecularly engineered live-attenuated chimeric West Nile/dengue virus vaccines protect rhesus monkeys from West Nile virus. Virology 2003 Sep. 15; 314(1):190-5.
[10] Pletnev A G, Swayne D E, Speicher J, Rumyantsev A A, Murphy B R. Chimeric West Nile/dengue virus vaccine candidate: preclinical evaluation in mice, geese and monkeys for safety and immunogenicity. Vaccine 2006 Sep. 29; 24(40-41):6392-404.
[11] Hanley K A, Goddard L B, Gilmore L E, Scott T W, Speicher J, Murphy B R, Pletnev A G. Infectivity of West Nile/dengue chimeric viruses for West Nile and dengue mosquito vectors. Vector Borne Zoonotic Dis 2005 Spring; 5(1):1-10.
[12] Tesh R B, da Rosa A P, Guzman H, araujo T P, Xiao S Y. Immunization with heterologous flaviviruses protective against fatal West Nile encephalitis. Emerg Inf Dis 2002; 8(3):245-51.
[13] Fang Y, Reisen W. Previous infection with West Nile or St. Louis encephalitis viruses provides cross protection during reinfection in house finches. Am J Trop Med Hyg 2006; 75(3):480-5.
[14] Hanley K A, Lee J J, Blaney J E, Jr., Murphy B R, Whitehead S S. Paired charge-to-alanine mutagenesis of dengue virus type 4 NS5 generates mutants with temperature-sensitive, host range, and mouse attenuation phenotypes. J Virol 2002 January; 76(2):525-31.
[15] Pletnev A G, Bray M, Huggins J, Lai C J. Construction and characterization of chimeric tick-borne encephalitis/dengue type 4 viruses. Proc Natl Acad Sci USA 1992 Nov. 1; 89(21):10532-6.
[16] Pletnev A G, Men R. Attenuation of the Langat tick-borne flavivirus by chimerization with mosquito-borne flavivirus dengue type 4. Proc Nati Acad Sci USA 1998; 95(4): 1746-51.
[17] Blaney J E, Manipon G G, Firestone C Y, Johnson D H, Hanson CT, Murphy B R, Whitehead S S. Mutations which enhance the replication of dengue virus type 4 and an antigenic chimeric dengue virus type 2/4 vaccine candidate in Vero cells. Vaccine 2003 Oct. 1; 21(27-28):4317-27.
[18] Monath T P, Soike K, Levenbook I, Zhang Z X, Arroyo J, Delagrave S, Myers G, Barrett A D, Shope R E, Ratterree M, Chambers T J, Guirakhoo F. Recombinant, chimaeric live, attenuated vaccine (ChimeriVax) incorporating the envelope genes of Japanese encephalitis (SA14-14-2) virus and the capsid and nonstructural genes of yellow fever (17D) virus is safe, immunogenic and protective in non-human primates. Vaccine 1999 Apr. 9; 17(15-16):1869-82.
[19] Rumyantsev A A, Chanock R M, Murphy B R, Pletnev A G. Comparison of live and inactivated tick-borne encephalitis virus vaccines for safety, immunogenicity and efficacy in rhesus monkeys. Vaccine 2006 Jan. 12; 24(2):133-43.
[20] Whitehead S S, Hanley K A, Blaney J E, Gilmore L E, Elkins W R, Murphy B R. Substitution of the structural genes of dengue virus type 4 with those of type 2 results in chimeric vaccine candidates which are attenuated for mosquitoes, mice, and rhesus monkeys. Vaccine 2003 Oct. 1; 21(27-28):4307-16.
[21] Pletnev A G, Bray M, Hanley K A, Speicher J, Elkins R. Tick-borne Langat/mosquito-borne dengue flavivirus chimera, a candidate live attenuated vaccine for protection against disease caused by members of the tick-borne encephalitis virus complex: evaluation in rhesus monkeys and in mosquitoes. J Virol 2001 September; 75(17):8259-67.
[22] Bray M, Lai C J. Construction of intertypic chimeric dengue viruses by substitution of structural protein genes. Proc Natl Acad Sci USA 1991;88(22):10342-6.
[23] Guirakhoo F, Weltzin R, Chambers T J, Zhang Z X, Soike K, Ratterree M, Arroyo J, Georgakopoulos K, Catalan J, Monath T P. Recombinant chimeric yellow fever-dengue type 2 virus is immunogenic and protective in nonhuman primates. J Virol 2000; 74(12):5477-85.
[24] Huang C Y, Butrapet S, Tsuchiya K R, Bhamarapravati N, Gubler D J, Kinney R M. Dengue 2 PDK-53 virus as a chimeric carrier for tetravalent dengue vaccine development. J Virol 2003 Nov. 1; 77(21):11436-47.
[25] Chambers T J, Jiang X, Droll D A, Liang Y, Wold W S, Nickells J. Chimeric Japanese encephalitis virus/dengue 2 virus infectious clone: biological properties, immunogenicity and protection against dengue encephalitis in mice. J Gen Virol 2006 November; 87(Pt 11):3131-40.
[26] Pugachev K V, Guirakhoo F, Mitchell F, Ocran S W, Parsons M, Johnson B W, Kosoy O L, Lanciotti R S, Roehrig J T, Trent D W, Monath T P. Construction of yellow fever/St. Louis encephalitis chimeric virus and the use of chimeras as a diagnostic tool. Am J Trop Med Hyg 2004; 71(5):639-45.
[27] Monath T P, Guirakhoo F, Nichols R, Yoksan S, Schrader R, Murphy C, Blum P, Woodward S, McCarthy K, Mathis D, Johnson C, Bedford P. Chimeric live, attenuated vaccine against Japanese encephalitis (ChimeriVax-JE): phase 2 clinical trials for safety and immunogenicity, effect of vaccine dose and schedule, and memory response to challenge with inactivated Japanese encephalitis antigen. J Infect Dis 2003;188(9):1213-30.
[28] Durbin A P, McArthur J H, Marron J A, Blaney J E, Thumar B, Wanionek K, Murphy B R, Whitehead S S. rDEN2/4Delta30(ME), A Live Attenuated Chimeric Dengue Serotype 2 Vaccine Is Safe and Highly Immunogenic in Healthy Dengue-Naive Adults. Hum Vaccines 2006 Nov. 5; 2(6):255-60.
[29] Guirakhoo F, Kitchener S, Morrison D, Forrat R, McCarthy K, Nichols R, Yoksan S, Duan X, Ermak T H, Kanesa-Thasan N, Bedford P, Lang J, Quentin-Millet M J, Monath T P. Live attenuated chimeric yellow fever dengue type 2 (ChimeriVax-DEN2) vaccine: Phase I clinical trial for safety and immunogenicity: effect of yellow fever pre-immunity in induction of cross neutralizing antibody responses to all 4 dengue serotypes. Human Vaccines 2006 March-April; 2(2):60-7.
[30] Monath T P, Liu J, Kanesa-Thasan N, Myers G A, Nichols R, Deary A, McCarthy K, Johnson C, Ermak T, Shin S, Arroyo J, Guirakhoo F, Kennedy J S, Ennis F A, Green S, Bedford P. A live, attenuated recombinant West Nile virus vaccine. Proc Natl Acad Sci USA 2006 Apr. 25; 103(17):6694-9.
[31] Wright P F, Ankrah S, Henderson S E, Durbin A P, Speicher J, Whitehead S S, Murphy B R, Pletnev A G. Evaluation of the Langat/dengue 4 chimeric virus as a live attenuated tick-borne encephalitis vaccine for safety and immunogenicity in healthy adult volunteers. Vaccine 2008; 26(7):882-90.
[32] Blaney J E, Jr., Hanson C T, Firestone C Y, Hanley K A, Murphy B R, Whitehead S S. Genetically modified, live attenuated dengue virus type 3 vaccine candidates. Am J Trop Med Hyg 2004; 71(6):811-21.
[33] Pletnev A G, Bray M, Lai C J. Chimeric tick-borne encephalitis and dengue type 4 viruses: effects of mutations on neurovirulence in mice. J Virol 1993 August; 67(8):4956-63.
[34] Blaney J E, Jr., Sathe N S, Hanson C T, Firestone C Y, Murphy B R, Whitehead S S. Vaccine candidates for dengue virus type 1 (DEN1) generated by replacement of the structural genes of rDEN4 and rDEN4Delta30 with those of DEN1. Virol J 2007; 4:23.
[35] Chambers T J, Nestorowicz A, Mason P W, Rice C M. Yellow fever/Japanese encephalitis chimeric viruses: construction and biological properties. J Virol 1999; 73(4):3095-101.
[36] Huang C Y, Butrapet S, Pierro D J, Chang G J, Hunt A R, Bhamarapravati N, Gubler D J, Kinney R M. Chimeric dengue type 2 (vaccine strain PDK-53)/dengue type 1 virus as a potential candidate dengue type 1 virus vaccine. J Virol 2000; 74(7):3020-8.
[37] Durbin A P, Karron R A, Sun W, Vaughn D W, Reynolds M J, Perreault J R, Thumar B, Men R, Lai C J, Elkins W R, Chanock R M, Murphy B R, Whitehead S S. Attenuation and immunogenicity in humans of a live dengue virus type-4 vaccine candidate with a 30 nucleotide deletion in its 3′-untranslated region. Am J Trop Med Hyg 2001; 65(5):405-13.
[38] Blaney J E, Jr., Durbin A P, Murphy B R, Whitehead S S. Development of a live attenuated dengue virus vaccine using reverse genetics. Viral Immunol 2006 Spring; 19(1):10-32.
[39] Maximova O A, Ward J M, Asher D M, St Claire M, Finneyfrock B W, Speicher J M, Murphy B R, Pletnev A G. Comparative neuropathogenesis and neurovirulence of attenuated flaviviruses in non-human primates. J Virol 2008; in press.
[40] Vorndam V, Mathews J H, Barrett A D, Roehrig J T, Trent D W. Molecular and biological characterization of a non-glycosylated isolate of St Louis encephalitis virus. J Gen Virol 1993; 74(Pt 12):2653-60.
[41] Chambers T J, Halevy M, Nestorowicz A, Rice C M, Lustig S. West Nile virus envelope proteins: nucleotide sequence analysis of strains differing in mouse neurovirulence. J Gen Virol 1998; 79:2375-80.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby expressly incorporated by reference in their entireties.

1. A nucleic acid comprising a first nucleotide sequence encoding at least one structural protein from a SLE virus and a second nucleotide sequence encoding at least one nonstructural protein from a dengue virus.

2. The nucleic acid of claim 1, wherein said at least one non-structural protein from a dengue virus comprises at least NS5.

3. The nucleic acid of claim 2, wherein said NS5 protein comprises at least one mutation.

4. The nucleic acid of claim 3, wherein said at least one mutation is a substitution at position 654 or 655 or a corresponding position when said dengue virus is not DEN4 or DEN4Δ30.

5. The nucleic acid of claim 1, wherein said at least one structural protein from a SLE virus is the E protein.

6. The nucleic acid of claim 5, wherein said E protein comprises at least one mutation.

7. The nucleic acid of claim 6, wherein said at least one mutation in the E protein is a substitution at position 156 or a corresponding position when said SLE virus is not the Hubbard strain of SLE.

8. The nucleic acid of claim 1, wherein said E protein comprises at least one mutation.

9. The nucleic acid of claim 8, wherein said at least one mutation in the E protein is at position 156 or a corresponding position when said SLE virus is not the Hubbard strain of SLE.

10. The nucleic acid of claim 1, wherein the dengue virus is dengue type 1 virus.

11. The nucleic acid of claim 1, wherein the dengue virus is dengue type 2 virus.

12. The nucleic acid of claim 1, wherein the dengue virus is dengue type 3 virus.

13. The nucleic acid of claim 1, wherein the dengue virus is dengue type 4 virus.

14. The nucleic acid nucleic acid of claim 1, wherein the dengue virus is an attenuated virus or a virus adapted for enhanced replication in Vero cells.

15. The nucleic acid of claim 1, wherein the dengue virus is dengue type 4 virus and the virus is attenuated by a deletion of about 30 nucleotides from the 3′ untranslated region of the dengue type 4 genome corresponding to the TL2 stem-loop structure between about nucleotides 10478-10507.

16. The nucleic acid of claim 1, wherein the dengue virus is dengue type 1 virus and the virus is attenuated by a deletion of about 30 nucleotides from the 3′ untranslated region of the dengue type 1 genome corresponding to the TL2 stem-loop structure between about nucleotides 10562-10591.

17. The nucleic acid of claim 1, wherein the dengue virus is dengue type 2 virus and the virus is attenuated by a deletion of about 30 nucleotides from the 3′ untranslated region of the dengue type 2 genome corresponding to the TL2 stem-loop structure between about nucleotides 10541-10570.

18. The nucleic acid of claim 1, wherein the dengue virus is dengue type 3 virus and the virus is attenuated by a deletion of about 30 nucleotides from the 3′ untranslated region of the dengue type 3 genome corresponding to the TL2 stem-loop structure between about nucleotides 10535-10565.

19. The nucleic acid of claim 1, wherein the first nucleotide sequence encodes at least two structural proteins from a SLE virus.

20. The nucleic acid of claim 1, wherein the structural proteins from a SLE virus are prM and E proteins.

21. The nucleic acid of claim 1, wherein said nucleic acid is selected from the group consisting of SEQ. ID. NOs.: 5, 7, 9, 11, and 13 or a fragment thereof, which contains at least a first nucleotide sequence encoding at least one structural protein from a SLE virus and a second nucleotide sequence encoding at least one nonstructural protein from a dengue virus.

22. A polypeptide encoded by any one or more of the nucleic acids set forth in claim 1.

23. The polypeptide of claim 22, wherein said polypeptide comprises the sequence of SEQ. ID. NOS.: 6, 8, 10, 12, or 14.

24. A chimeric virus comprising any one or more of the nucleic acids or polypeptides set forth in claim 1.

25. An immunogenic composition comprising any one or more of the nucleic acids or polypeptides set forth in claim 1.

26. The immunogenic composition of claim 25 for use in the induction of an immune response.

27. A method of inducing an immune response in a subject comprising: providing an effective amount of the composition of claim 25 to the subject; and
measuring the induction of an immune response in said subject.

28. A vaccine comprising any one or more of the nucleic acids or polypeptides set forth in claim 1.

29. A method of providing protection against SLE infection in a subject comprising:
identifying a subject in need of protection against SLE infection; and
providing an effective amount of the composition of claim 25 to the subject.
